(19) United States
(12) Patent Application Publication
(10) Pub. No.: US 2025/0082333 A1
(43) Pub. Date: Mar. 13, 2025

(54) LUMEN ABLATION SYSTEM AND METHOD OF TREATING VARICOSE VEINS WITH SAME

(71) Applicant: Kevin T. LIE, Greenville, DE (US)

(72) Inventor: Kevin T. LIE, Greenville, DE (US)

(21) Appl. No.: 18/960,802

(22) Filed: Nov. 26, 2024

Related U.S. Application Data
(63) Continuation-in-part of application No. PCT/US2023/023647, filed on May 26, 2023.
(60) Provisional application No. 63/346,078, filed on May 26, 2022.
(60) Provisional application No. 63/602,639, filed on Nov. 26, 2023.

(51) Int. Cl. A61B 17/12

(52) U.S. Cl. CPC A61B 17/12109; A61B 17/12136

(57) ABSTRACT

A lumen ablation system provides a method of treating diseased veins, such as varicose veins with a pair of balloons to isolate a section of lumen. A toroid access balloon is configured proximal to a lumen access aperture. An isolation balloon is configured an offset distance from the access balloon to form an isolated lumen between the two inflated balloons. The isolation balloon may be translated along a guide wire that extends through an access port and also through the toroid access balloon aperture. An ablation treatment apparatus with an ablation implement, such as an ablation brush or tines is translated into the isolated lumen. The ablation treatment apparatus may be configured on a catheter having a conduit for aspirating the lumen with therapeutic agent. The isolated lumen may be ablated by moving the ablation treatment apparatus within the therapeutic agent, such as a sclerosant therein.

### CROSS REFERENCE TO RELATED APPLICATIONS

This application is a bypass continuation-in-part of PCT Application No. PCT/US2023/023647, filed May 26, 2023, which claims the benefit of priority to U.S. Provisional Patent Application No. 63/346,078, filed on May 26, 2022, and this application also claims the benefit of U.S. Provisional Application No. 63/602,639, filed Nov. 26, 2023; the entirety of all prior application are hereby incorporated by reference herein.

### BACKGROUND OF THE INVENTION

### Field of the Invention

The invention relates to a lumen ablation system and method of treating varicose veins with said lumen ablation system.

### Background

Varicose veins are typically treated with a sclerosant that is injected into the vein and/or by ablation. The sclerosant is not isolated within the lumen and therefore can be reduced in concentration in a desired section of the lumen. Also, ablation can lead to debris that can travel along the lumen and cause clotting.

### SUMMARY OF THE INVENTION

The invention is directed to a lumen treatment system to infuse medication including, such thrombolysis, chemotherapy, and./or for ablation of the lumen. A portion of the lumen may be isolated for thrombolysis for example or for ablation. The lumen treatment system may be employed in a method of treating varicose veins via lumen ablation.

An exemplary lumen treatment system may infuse medication for disruption and removal of clots, or thrombolysis. Thrombolytic agents included, but are not limited to, Eminase (anistreplase) Retavase (reteplase) Streptase (streptokinase, kabikinase), Alteplase, Retaplase, Tenacteplase, urokinase, prourokinase, and anistreeplase (APSAC). Other medications that may be introduced intraarterially through the lumen treatment system include, BCNU, cisplatin, etoposide, methotrexate, and carboplatin, that are some of the drugs that have been used intra-arterially to treat PBT and MBT.

An exemplary lumen treatment system may infuse medication into a lumen other than an arterial lumen, such as a lumen of the digestive or respiratory system. Medications may be used to treat ulcers, tumors, and other conditions within the respective system.

An exemplary lumen treatment system may be a lumen ablation system that utilizes a pair of balloons to isolate a section of lumen for treatment. An access balloon that is a toroid balloon with an access balloon aperture is configured proximal to a lumen access aperture. An isolation balloon is configured an offset distance from the access balloon to form an isolated lumen between the two inflated balloons. The isolation balloon may be translated along a guide wire that extends through an access port and also through the access balloon aperture. An ablation treatment apparatus is translated into the isolated lumen through the access port and also through the access balloon aperture. The ablation treatment apparatus has an ablation implement, such as an ablation brush comprising a plurality of tines extending radially outward from the ablation catheter, forming an ablation conduit or agent conduit. The ablation treatment apparatus may be configured on a distal end of a catheter and the catheter may provide a conduit for aspirating the isolated lumen and/or delivering therapeutic agent into the isolated lumen. The isolated lumen may be ablated by moving the ablation treatment apparatus back and forth with the therapeutic agent, such as a sclerosant therein.

An exemplary access port is configured through a lumen access aperture and is configured over a guide wire that extends through the access port conduit and into the lumen. The access to the lumen may be created by first puncturing the lumen wall with a needle. An introducer sheath may then be configured down over the needle and into the lumen. An exemplary introducer sheath has a needle conduit to enable the introducer sheath to translate over the needle. Also, an exemplary introducer sheath has a guide wire deflector with opposing surfaces that are configured to direct the guide wire in either direction along the lumen. The guide wire deflector may be a generally dome shaped deflector that extends up into the introducer sheath conduit. A pair of guide wire apertures may be configured to align with the opposing deflecting surfaces of the guide wire deflector. The opposing surfaces of the guide wire deflector may be curved surfaces and may be concave curved surfaces. Fluoroscopy may be used to ensure that the guide wire is directed in a desired direction along the lumen.

An exemplary access balloon may be coupled on a distal end of the access port and an access balloon inflation conduit may extend along the access port to an access balloon interface for inflating and deflating the access balloon. The access balloon, access balloon inflation conduit and/or the access balloon interface may be part of the access port. The access balloon is a toroid balloon having a toroid shape with a access balloon aperture therethrough to allow introduction and removal of the guide wire, insolation balloon and ablation treatment apparatus therethrough. An access port conduit may be in fluid communication with the access balloon aperture to enable a smooth interface for the introduction of components into the lumen.

An isolation balloon may be an over-wire balloon that extends around the guide wire and the guide wire may be the isolation balloon inflation conduit. An isolation balloon inflation conduit may be coupled directly with the isolation balloon and may act as a shaft to push the isolation balloon along the guide wire to an offset distance from the access balloon. The isolation balloon conduit may be coupled with an isolation balloon interface to enable introduction of an inflation fluid into the isolation balloon to inflate the isolation balloon. The isolation balloon may be configured at an offset angle to the length axis of the guide wire or isolation balloon inflation conduit to enable steering the isolation balloon to a desired location within the lumen.

An exemplary ablation treatment apparatus comprises ablation aperture to allow a flow of therapeutic agent into the lumen, such as sclerosant. The ablation treatment apparatus may have one or more apertures through the ablation conduit wall, or apertures that are radial to the length of the ablation conduit. Also, an ablation aperture may be configured in a distal end of the ablation conduit. The location of the ablation apertures with respect to the ablation implement may prevent sealing off of the ablation apertures during aspiration of the lumen. The agent apertures in the ablation treatment apparatus may be configured in the ablation conduit wall distal from the ablation implement or between tines of the ablation implement to prevent the lumen from collapsing down over the agent apertures.

The ablation treatment apparatus may be on a distal end of an ablation catheter to allow aspiration of the lumen and also introduction of the therapeutic agent into the lumen via an ablation interface.

An exemplary ablation treatment apparatus has an ablation implement configured to ablate or injure the inside surface of the lumen wall by scraping along the inside surface of the lumen wall. An exemplary ablation implement comprises a plurality of tines that extend radially outward from the ablation conduit, thereby forming an ablation brush. The individual tines may enable the isolation balloon inflation conduit to move between the tines as the ablation implement is moved back and forth to ablate the lumen.

The lumen may be aspirated to cause the lumen to collapse, which may increase the effectiveness of the physical ablation via the ablation implement. The access port and/or the isolation balloon inflation conduit through the ablation interface may be used to aspirate the lumen initially and then the ablation treatment apparatus may be used to aspirate the lumen and may be moved between the access balloon and isolation balloon to provide effective aspiration and also collapse of the lumen.

The therapeutic agent may be introduced into the lumen and retained between the isolation balloon and the access balloon. The ablation implement may be moved back and forth within the isolated lumen after the introduction of the therapeutic agent. The lumen may be aspirated after introduction of the therapeutic agent and after ablation to remove the therapeutic agent and any debris from the ablation process.

An exemplary lumen treatment system may be used to treat and, in some cases, collapse a portion of the saphenous vein. Also, the lumen treatment system may be used to treat and/or ablate and collapse one or more of the perforating veins extending between the saphenous vein and the femoral vein. A single access may be utilized to introduce the integrated isolation ablation component into the saphenous vein and into the perforating veins. A guide extension may be utilized to direct or steer the guide wire into the saphenous vein and also into a perforating vein. In an exemplary method, a portion of the saphenous vein more proximal the heart is treated and collapsed before the guide wire is directed into a perforating vein, such as the more proximal perforating vein to the heart. The guide wire may extend through the perforating vein and into the femoral vein. The isolation balloon may be inflated to seal the perforating vein from the femoral vein and may be configured at or near the intersection of the perforating vein with the femoral vein, or proximal the distal end of the perforating vein. With the perforating vein isolated from the femoral vein and also within the isolated vein region, the integrated isolation ablation component may be advanced into the perforating vein for ablation, therapeutic agent introduction and the application of vacuum to remove the therapeutic agent and collapse the perforating vein.

An exemplary lumen treatment system may be used to treat and in some cases collapse or ablate a portion of a gonadal vein. In medicine, gonadal vein refers to the blood vessel that carries blood away from the gonad (testis, ovary) toward the hearts pelvic congestion syndrome and may be an ovarian vein or testicular vein. Blood flow from the uterus, ovaries and fallopian tubes flows through the ovarian veins to a respective left and right renal vein. When the ovarian veins are diseased it can result in a pain, such as from pelvic congestion syndrome. A left ovarian vein and right ovarian vein are coupled with a respective ovarian venous plexus. Testicular veins extend from respective testicles to the left and right renal veins. A lumen ablation system, as described herein, may be used to ablate the left and/or right gonadal vein. A guide extension may direct the guide wire and isolation balloon into one of gonadal veins, such as from the inferior vena cava, but could be introduced from a renal vein, such as the left renal vein or even along the left ovarian vein.

A lumen, as used herein, may be a vasculature lumen, such as a vein or artery, or other lumen within the body such as any lumen in the digestive track including intestine, urethra and the like.

The summary of the invention is provided as a general introduction to some of the embodiments of the invention, and is not intended to be limiting. Additional example embodiments including variations and alternative configurations of the invention are provided herein.

### BRIEF DESCRIPTION OF SEVERAL VIEWS OF THE DRAWINGS

The accompanying drawings are included to provide a further understanding of the invention and are incorporated in and constitute a part of this specification, illustrate embodiments of the invention, and together with the description serve to explain the principles of the invention.

FIG. 1 shows a side cross sectional view of a lumen, such as a vein, and a lumen ablation system with an ablation treatment apparatus configured between an access balloon and an isolation balloon to treat the inside of the isolated lumen wall with therapeutic agent and with a physical ablation implement, such as a brush.

FIG. 2 shows a side view of an introducer sheath for a guide wire having a deflector to enable the guide wire to be guided in opposing directions along the lumen.

FIG. 3 shows a front view of the introducer sheath shown in FIG. 2 having a guide wire access aperture in the wall of the introducer sheath with the deflector configure within and extending up from the distal end of the introducer sheath.

FIG. 4 shows an introducer sheath as depicted in FIGS. 2 and 3 now configured through a lumen access aperture in the lumen wall of a lumen, such as a vein, and a guide wire being directed in a first direction along the lumen.

FIG. 5 shows the introducer sheath shown in FIG. 4 but with the guide wire now being deflected by the guide wire deflector in a second direction along the lumen, opposite the first direction shown in FIG. 4.

FIG. 6 shows an access port configured in a lumen access aperture in the lumen wall with an access balloon, a toroid balloon, configured on the distal end of the access port with an access balloon aperture for introducing a guide wire, an isolation balloon and also the ablation treatment apparatus therethrough.

FIG. 7 shows the lumen ablation system shown in FIG. 6, with the access balloon inflated by the access-balloon interface and the guide wire being introduced into the lumen through the access port, and the access balloon aperture.

FIG. 8 shows the lumen ablation system shown in FIG. 7, with an isolation balloon, an over-wire balloon, being inflated by the isolation-balloon interface.

FIG. 9 shows the lumen ablation system shown in FIG. 8, with the isolation balloon now inflated to produce an isolated lumen between the isolation balloon and the access balloon.

FIG. 10 shows the lumen ablation system shown in FIG. 9, with the access interface aspirating the isolated lumen to remove the blood from the isolated lumen.

FIG. 11 shows the lumen ablation system shown in FIG. 10, with the ablation treatment apparatus configured over the guide wire and being introduced through the access port.

FIG. 12 shows the lumen ablation system shown in FIG. 11, with the ablation treatment apparatus introduced into the isolated lumen through the access port and aspirating the isolated lumen to further collapse the lumen through the ablation interface.

FIG. 13 shows the lumen ablation system shown in FIG. 12 with the ablation treatment apparatus further introduced toward the isolation balloon and aspirating to collapse the lumen down between the isolation balloon and the access balloon.

FIG. 14 shows the lumen ablation system shown in FIG. 13, with the ablation treatment apparatus introducing therapeutic agent through the therapeutic agent conduit and out of the ablation treatment apparatus into the isolated lumen.

FIG. 15 shows the lumen ablation system shown in FIG. 14, with the ablation treatment apparatus being translated back and forth within the isolated lumen to ablate the lumen having therapeutic agent therein.

FIG. 16 shows the lumen ablation system shown in FIG. 15, with vacuum being drawn through the ablation treatment apparatus to remove therapeutic agent and any debris within the isolated lumen and to collapse the isolated lumen.

FIG. 17 shows the lumen ablation system shown in FIG. 16, with the ablation treatment apparatus withdrawn from the lumen and the isolated lumen collapsed.

FIG. 18 shows the lumen ablation system shown in FIG. 17, with the isolation balloon collapsed for removal.

FIG. 19 shows the lumen ablation system shown in FIG. 18, with the isolation balloon removed and the isolated lumen collapsed.

FIG. 20 shows a side cross sectional view of a lumen, such as a vein, and a lumen ablation system with an ablation treatment apparatus configured between an access balloon and an isolation balloon to treat the inside of the isolated lumen wall with therapeutic agent and with a physical ablation implement, such as a brush.

FIG. 21 shows an access port configured in a lumen access aperture in the lumen wall with an access balloon, a toroid balloon, configured on the distal end of the access port with an access balloon aperture for introducing a guide wire, an isolation balloon and also the ablation treatment apparatus therethrough.

FIG. 22 shows the lumen ablation system shown in FIG. 21, with the access balloon inflated by the access-balloon interface and the isolation ballon inflation conduit being introduced into the lumen through the access port, and the access balloon aperture.

FIG. 23 shows the lumen ablation system shown in FIG. 21, with an isolation balloon being inflated by the isolation balloon inflation conduit via the isolation-balloon interface.

FIG. 24 shows the lumen ablation system shown in FIG. 23, with the isolation balloon now inflated to produce an isolated lumen between the isolation balloon and the access balloon and the ablation treatment apparatus being inserted over the isolation balloon inflation conduit into the lumen.

FIG. 25 shows the lumen ablation system shown in FIG. 24, with the access interface aspirating the isolated lumen to remove the blood from the isolated lumen.

FIG. 26 shows the lumen ablation system shown in FIG. 25, with the ablation treatment apparatus being advanced into the isolated lumen through the access port and aspirating the isolated lumen to further collapse the lumen through the ablation interface.

FIG. 27 shows the lumen ablation system shown in FIG. 26 with the ablation treatment apparatus further introduced toward the isolation balloon and aspirating to collapse the lumen down between the isolation balloon and the access balloon.

FIG. 28 shows the lumen ablation system shown in FIG. 27, with the ablation treatment apparatus introducing therapeutic agent through the therapeutic agent conduit and out of the ablation treatment apparatus into the isolated lumen.

FIG. 29 shows the lumen ablation system shown in FIG. 28, with the ablation treatment apparatus being translated back and forth within the isolated lumen to ablate the lumen having therapeutic agent therein.

FIG. 30 shows the lumen ablation system shown in FIG. 29, with vacuum being drawn through the ablation treatment apparatus to remove therapeutic agent and any debris within the isolated lumen and to collapse the isolated lumen.

FIG. 31 shows the lumen ablation system shown in FIG. 30, with the ablation treatment apparatus withdrawn from the lumen and the isolated lumen collapsed.

FIG. 32 shows the lumen ablation system shown in FIG. 31, with the isolation balloon collapsed for removal.

FIG. 33 shows the lumen ablation system shown in FIG. 32, with the isolation balloon removed and the isolated lumen collapsed.

FIG. 34 shows a person's leg and the anatomy of the femoral vein, the saphenous vein and the perforating veins.

FIG. 35 shows an expanded view of a healthy vascular system with the blood flow in the femoral vein and saphenous vein flowing from the lower extremity to the heart and blood flow from the saphenous vein through the perforating vein to the femoral vein.

FIG. 36 shows an expanded view of a diseased vascular system with the blood flow in the femoral vein flowing from the lower extremity to the heart but the blood flow in the saphenous vein down toward the lower extremity and blood flow from the femoral vein through the perforating vein to the saphenous vein.

FIG. 37 shows a guide wire being advanced along the saphenous vein, and guided through the perforating vein into the femoral vein.

FIG. 38 shows a balloon expanded at the intersection of the femoral vein and the perforating vein to seal the perforating vein from the femoral vein.

FIG. 39 shows the integrated isolation ablation component advanced over the guide wire into the perforating vein.

FIG. 40 shows therapeutic agent being dispensed into the perforating vein and the therapeutic agent being blocked from flow into the femoral vein by the expanded balloon.

FIG. 41 shows the integrated isolation ablation component retracted from the perforating vein and suction being applied to collapse the perforating vein.

FIG. 42 shows the guide wire now advanced into the saphenous vein to treat and collapse the saphenous vein down.

FIG. 43 shows a lumen ablation system being introduced into the left ovarian vein, an example of a gonadal vein.

FIG. 44 shows a lumen ablation system configured to seal off the gonadal vein for ablation of said gonadal vein.

FIG. 45 shows a lumen ablation system configured to seal off the gonadal vein for ablation of said gonadal vein.

FIG. 46 shows a side view of the ablation treatment assembly, including an ablation treatment apparatus having an ablation implement that is configured circumferentially about the ablation catheter and specifically with the ablation implements extensions configured circumferentially aligned with at least one of the agent apertures that are configured on the distal or proximal ends of an ablation implement extension.

FIG. 47 shows a cross-section of the lumen ablation system taken along line 47 of FIG. 27 and shows the access port conduit and access balloon inflation conduit, the ablation catheter and the isolation balloon conduit.

FIG. 48 shows the isolation balloon assembly that is configured to extend through the ablation treatment assembly and through the ablation conduit to locate the isolation balloon distal the ablation treatment assembly.

FIG. 49 shows the ablation treatment assembly that is configured to extend through the access port conduit and into the vessel and having an ablation treatment apparatus with an ablation conduit for receiving therapeutic agent therein.

FIG. 50 shows an enlarged view of the ablation treatment apparatus having a plurality of agent apertures and ablation implements, such as tines extending from the ablation conduit radially outward to ablate an interior of lumen or vessel wall and wherein the agent apertures are configured in alignment with an ablation implement.

FIG. 51 shows the access port having an access port conduit extending from the access port inlet opening to the access port outlet opening and an access balloon, a toroid balloon, configured proximal to the distal end of the access port, and a lumen access interface, as well as a access balloon interface for inflating the access balloon.

FIG. 52 shows the lumen ablation system that includes the three components shown in FIGS. 48, 49 and 51 integrated together for isolating a segment of a lumen, such as a vessel, for ablating the vessel and collapsing the vessel.

Corresponding reference characters indicate corresponding parts throughout the several views of the figures. The figures represent an illustration of some of the embodiments of the present invention and are not to be construed as limiting the scope of the invention in any manner. Some of the figures may not show all of the features and components of the invention for ease of illustration, but it is to be understood that where possible, features and components from one figure may be included in the other figures. Further, the figures are not necessarily to scale, some features may be exaggerated to show details of particular components. Therefore, specific structural and functional details disclosed herein are not to be interpreted as limiting, but merely as a representative basis for teaching one skilled in the art to variously employ the present invention.

### DETAILED DESCRIPTION OF THE ILLUSTRATED EMBODIMENTS

As used herein, the terms “comprises,” “comprising,” “includes,” “including,” “has,” “having” or any other variation thereof, are intended to cover a non-exclusive inclusion. For example, a process, method, article, or apparatus that comprises a list of elements is not necessarily limited to only those elements but may include other elements not expressly listed or inherent to such process, method, article, or apparatus. Also, use of “a” or “an” are employed to describe elements and components described herein. This is done merely for convenience and to give a general sense of the scope of the invention. This description should be read to include one or at least one and the singular also includes the plural unless it is obvious that it is meant otherwise.

Certain exemplary embodiments of the present invention are described herein and are illustrated in the accompanying figures. The embodiments described are only for purposes of illustrating the present invention and should not be interpreted as limiting the scope of the invention. Other embodiments of the invention, and certain modifications, combinations and improvements of the described embodiments, will occur to those skilled in the art and all such alternate embodiments, combinations, modifications, improvements are within the scope of the present invention.

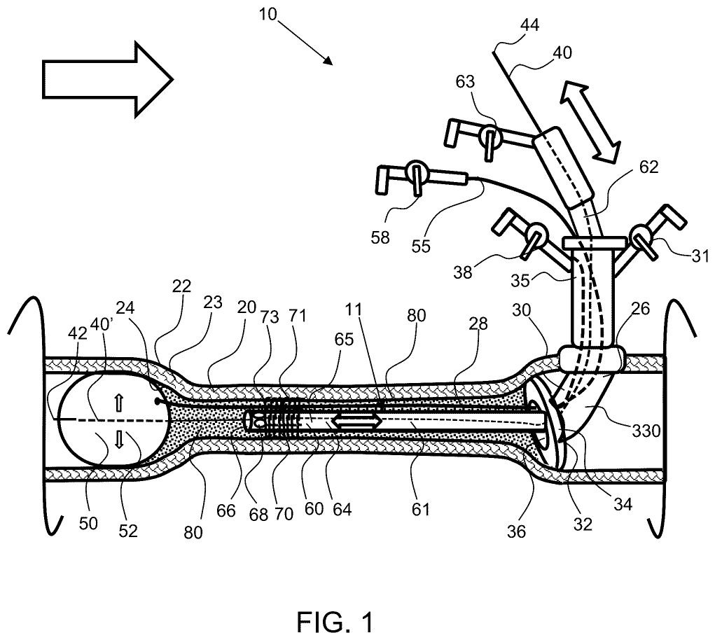

As shown in FIG. 1, an ablation treatment system 10 utilizes a lumen ablation apparatus 11 that is configured to isolate a portion of a lumen, an isolated lumen 28, between an access balloon 32 and an isolation balloon 50 to treat the interior surface 24 of the lumen wall 23 with therapeutic agent 80. The therapeutic agent 80 is delivered through the agent conduit to the ablation treatment apparatus 60 having agent apertures 68 to dispense the therapeutic agent into the isolated lumen 28. The ablation treatment apparatus 60 also has an ablation implement 70, such as an ablation brush 71 comprising a plurality of tines 73 that extend radially outward from the ablation conduit wall 65 and configured to physical ablate the interior surface 24 of the lumen wall 23 when the ablation treatment apparatus is moved back and forth within the isolated lumen 28. The therapeutic agent 80 is retained within the isolated lumen along with any plague that may be dislodged from the lumen wall during the ablation process.

The ablation treatment apparatus 60 and isolation balloon may be guided into the lumen by a guide wire 40 that extends through the access port 30, an access catheter 330, and the access balloon aperture 36 in the access balloon 32. The guide wire 40 extends through the ablation treatment apparatus 60, the access balloon aperture 36 in the access balloon 32 to a distal end 42 within the lumen 20. The isolation balloon 50 is guided along the guide wire 40 and is inflated by the introduction of inflation fluid through the isolation balloon inflation conduit 55 delivered by the inflation balloon interface 58. Inflation of the isolation balloon and the access balloon 32 produces the isolated lumen 28. The access balloon 32 is configured on the distal end of the access port 30 and is inflated by the introduction of inflation fluid through the access balloon inflation conduit 35 delivered by the access balloon interface 38 that may be part of the access port 30. The guide wire may be the isolation balloon conduit. The distal end 42 may be a steerable tip to enable the guide wire to be steered into a branch of a lumen.

The ablation treatment apparatus 60 is introduced through the access port 30, through the access balloon aperture 36 along the guide wire 40 and may be configured on or as part of an ablation catheter 61 that forms a therapeutic agent conduit 62 extending from the ablation treatment apparatus 60 to the ablation interface 63. The ablation interface may be used to move the ablation treatment apparatus 60 into and within the isolated lumen 28 and may also be used to aspirate the isolated lumen as well as deliver therapeutic agent, such as sclerosant into the isolated lumen. The therapeutic agent 80 is delivered to the ablation treatment apparatus 60 through the therapeutic agent conduit 62 by agent apertures 68 in the ablation treatment apparatus 60 via the therapeutic ablation interface 63, which may be a syringe or valve. The ablation treatment apparatus 60 may have agent apertures in the ablation conduit wall and also an end aperture on the distal end of the ablation treatment apparatus 60. The ablation implement 70, such as an ablation brush 71, is coupled with the ablation conduit 64 formed by an ablation conduit wall 65. The therapeutic agent 80 is delivered through agent apertures 68 in the ablation treatment apparatus 60, such as in the ablation conduit wall 65 or in the distal end of the ablation conduit 64. The ablation catheter has an end aperture 66 that may also allow agent to flow through.

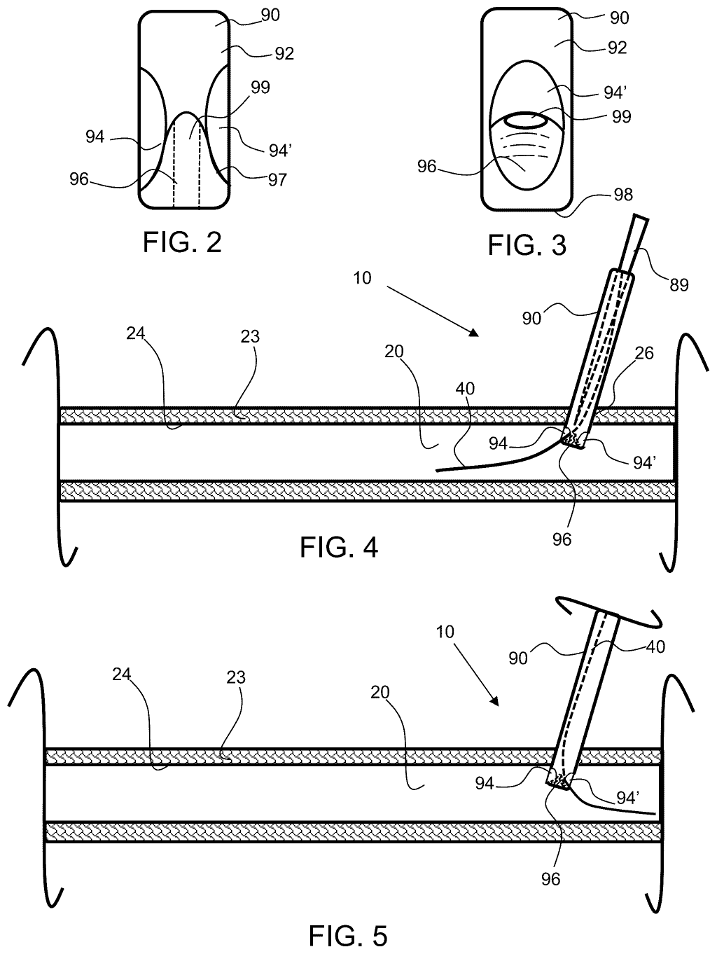

Referring now to FIGS. 2 to 5, access to the lumen may be initiated by a needle 89 that punctures through the lumen wall 23 from the exterior surface 22 to the interior surface 24 of the lumen wall. An introducer sheath 90 may then be configured over the needle and pushed through the lumen wall to provide access to the lumen 20. The needle may extend through the needle conduit 99 in the guide wire deflector 96, configured on the distal end 98 of introducer sheath 90. As shown in FIG. 2, the guide wire deflector 96 has opposing curved surfaces 97 that are configured to guide the guide wire 40 in opposing directions and out one of the guide wire apertures 94, 94′ in the introducer wall 92. As shown in FIG. 3, the needle conduit 99 is centrally configured in the guide wire deflector 96. As shown in FIG. 4, the guide wire 40 is being deflected by the guide wire deflector 96 and introduced through the guide wire aperture 94 in a first direction down the lumen 20 and as shown in FIG. 5 the guide wire 40 is being deflected by the guide wire deflector 96 and introduced through the guide wire aperture 94′ guided in a second direction down the lumen, opposite the first direction.

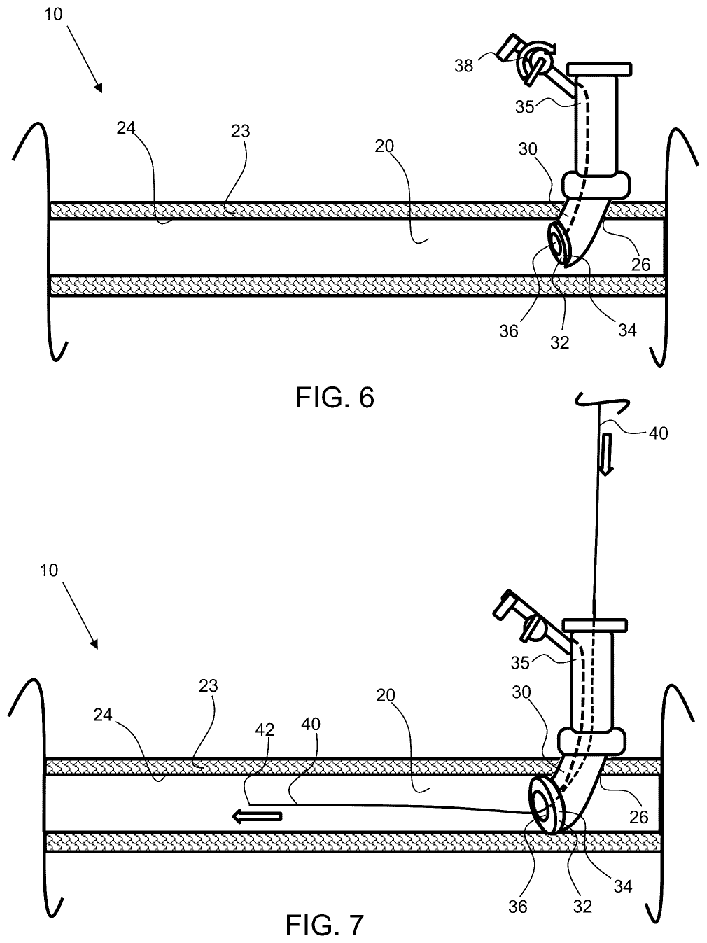

As shown in FIG. 6, an access port 30 is configured in a lumen access aperture 26 in the lumen wall 23 with an access balloon 32, a toroid balloon 34, configured on the distal end of the access port with an access balloon aperture 36 for introducing a guide wire, an isolation balloon and also the ablation treatment apparatus therethrough. The access balloon 32 is not inflated and does not occlude the lumen 20. The access balloon may be coupled to the access port 30 and the access port may include the access-balloon interface 38.

As shown in FIG. 7, the access balloon 32 has been inflated through the introduction of inflation fluid from the access balloon inflation conduit 35 and the access balloon interface 38. The lumen ablation system 10 has the distal end 42 of the guide wire 40 being introduced into the lumen 20 through the access port 30 and the access balloon aperture 36, in the access balloon 32. The access balloon has been inflated through the introduction of inflation fluid from the access balloon inflation conduit 35 and the access balloon interface 38. A syringe may be coupled with the access balloon interface to introduce the inflation fluid into the toroid balloon 34 forming the access balloon 32.

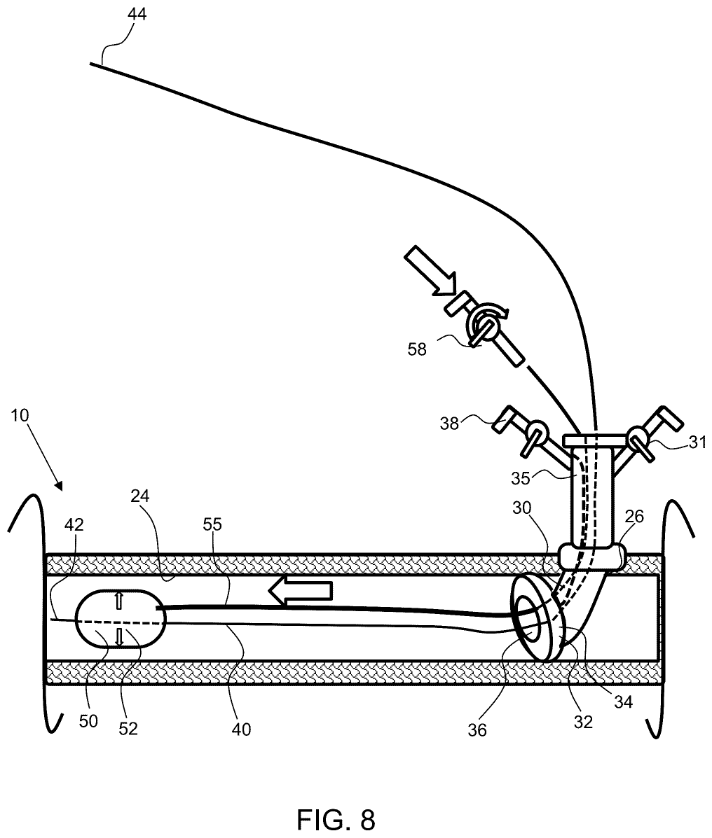

As shown in FIG. 8, the lumen ablation system 10 has the isolation balloon 50 introduced into the lumen 20 through the access port 30, and the access balloon aperture 36 in the access balloon 32. The isolation balloon 50 is being inflated by the introduction of an inflation fluid through the isolation balloon inflation conduit 55 via the isolation balloon interface 58. The isolation balloon inflation conduit may act as a shaft to push the inflation balloon 50 along the guide wire 40. The inflation balloon may be an over-wire balloon that extends over or around the guide wire. A syringe may be coupled with the isolation balloon interface to inject inflation fluid, such as saline or water, into the isolation balloon 50 to expand the isolation balloon wall 52 against the interior surface 24 of the lumen wall 23. The proximal end 44 of the guide wire 40 is shown.

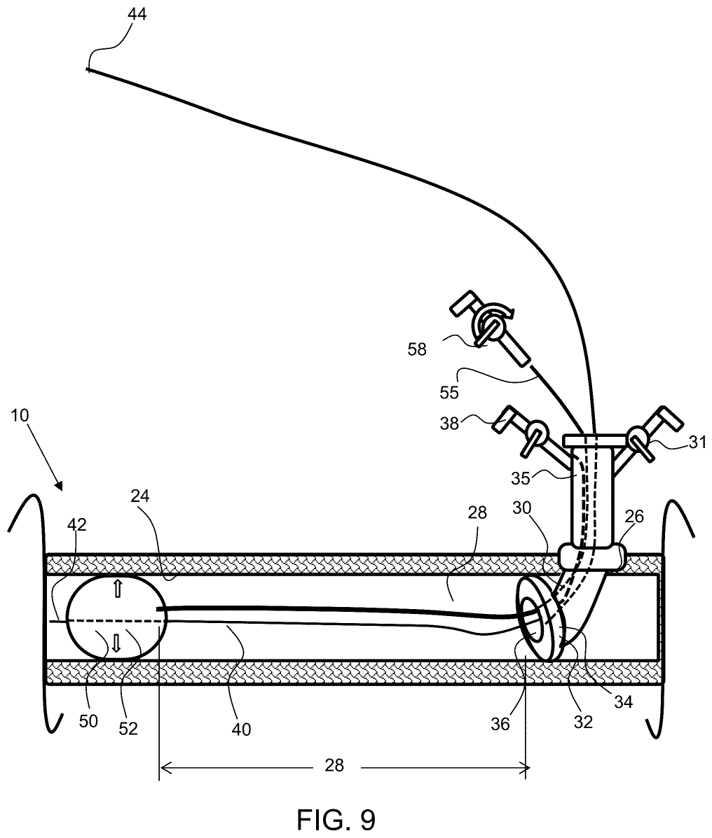

As shown in FIG. 9, the lumen ablation system 10 has the isolation balloon 50 now inflated to produce an isolated lumen 28 having a length 28 between the isolation balloon 50 and the access balloon 32. The isolated lumen will prevent therapeutic agent and thrombus or other debris from the ablation process from traveling along the lumen. The isolation balloon is configured an offset distance 28 from the access balloon 32 to create the isolated lumen 28.

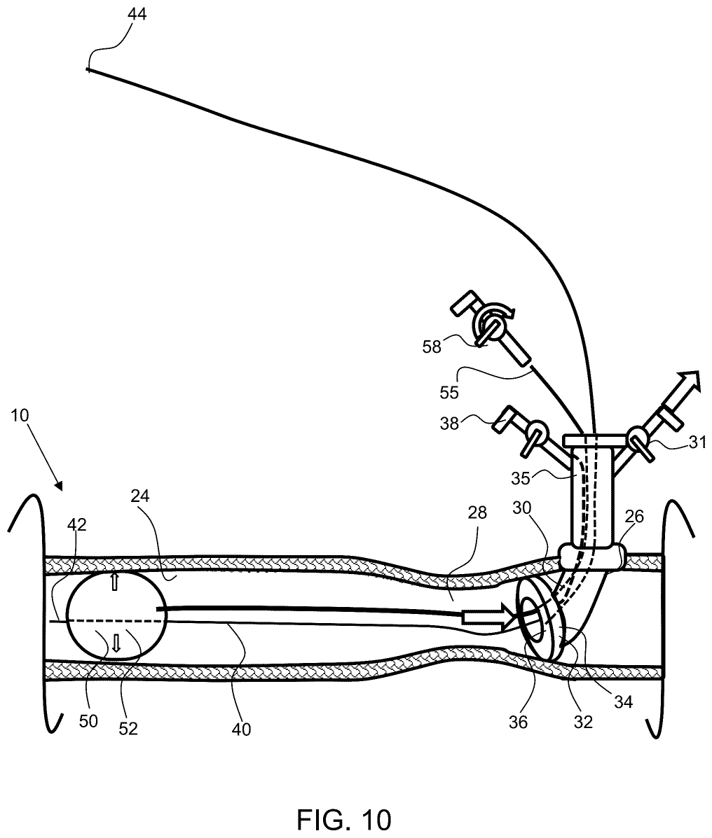

As shown in FIG. 10, the isolated lumen 28 is being aspirated by the lumen access interface 31, as indicated by the large bold arrow extending from the access balloon interface, to remove the blood from the isolated lumen.

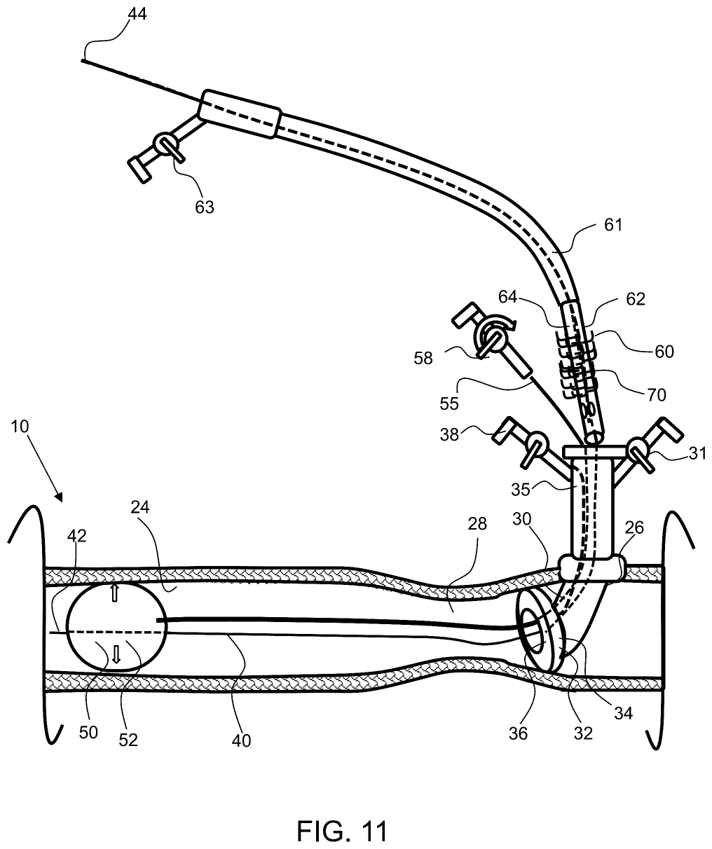

As shown in FIG. 11, the ablation treatment apparatus 60 is configured over the guide wire 40 and is being introduced through the access port 30.

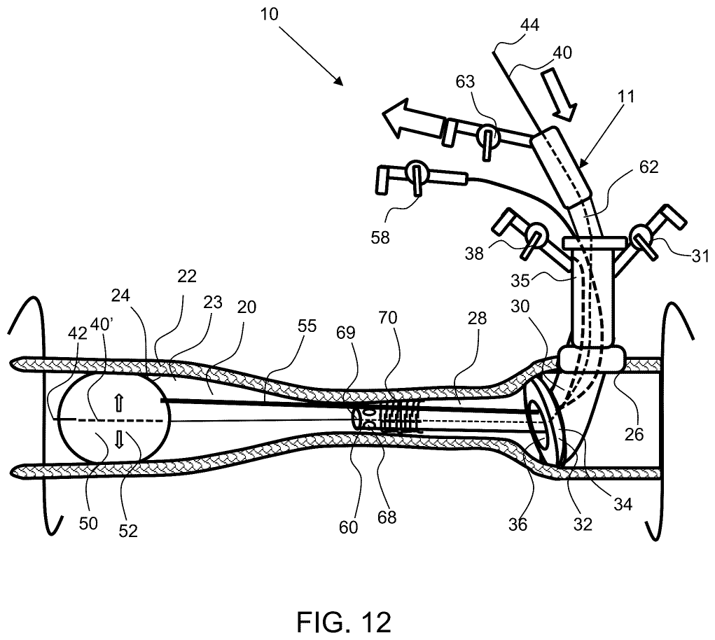

As shown in FIG. 12, the lumen ablation system 10 has the ablation treatment apparatus 60 introduced into the isolated lumen 28 through the access port 30 and through the access balloon aperture 36 in the access balloon 32. The isolated lumen is being further aspirated to remove any additional blood and to collapse the isolated lumen through the agent apertures in the ablation treatment apparatus 60. The ablation treatment apparatus may have an agent aperture on the distal end 69. The ablation treatment apparatus 60 may be moved to the isolation balloon and then withdrawn while the lumen is aspirated to remove blood and collapse the isolated lumen 28.

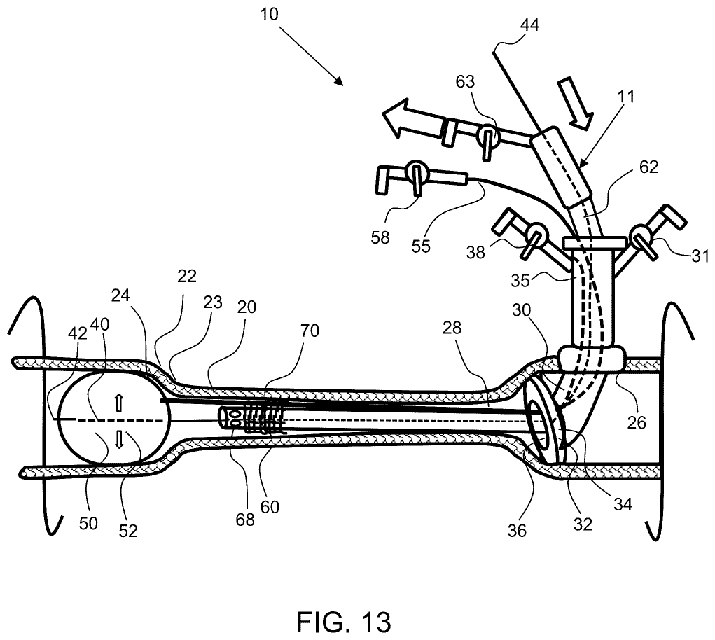

As shown in FIG. 13, the ablation treatment apparatus 60 is translated further toward the isolation balloon 50 and is aspirating the lumen 20 to cause it to collapse down between the isolation balloon and access balloon.

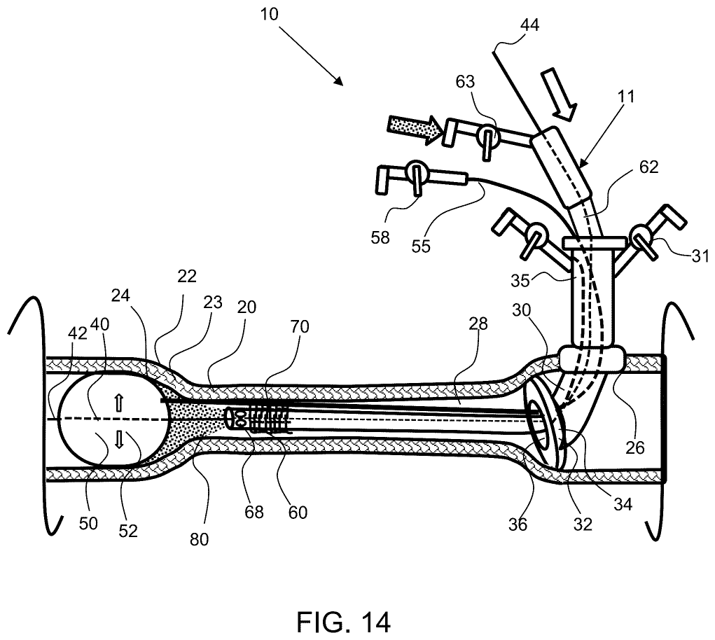

As shown in FIG. 14, therapeutic agent 80 is being introduced into the isolated lumen 28 by the ablation treatment apparatus 60 through the ablation interface 63 as indicated by the large arrow. The therapeutic agent 80 is being dispense from agent apertures 68 in the ablation treatment apparatus 60, such as in the ablation conduit wall 65 or in the distal end of the ablation conduit 64.

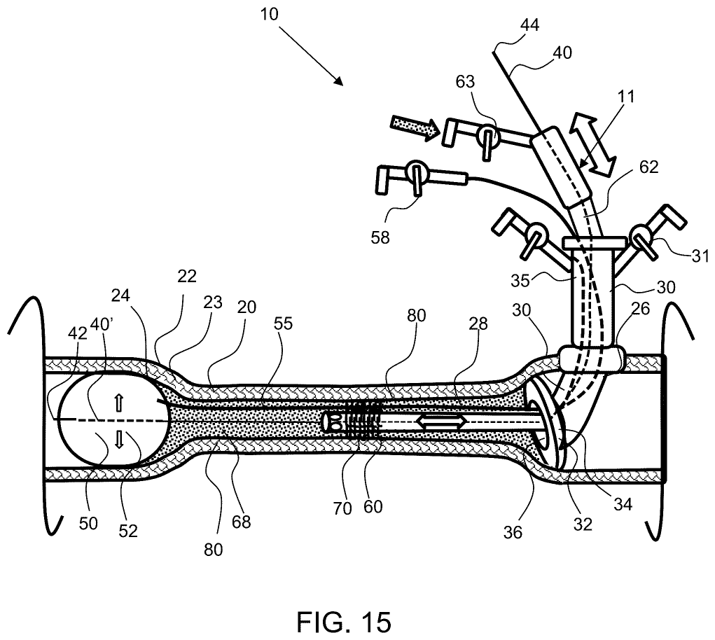

As shown in FIG. 15, the ablation treatment apparatus 60 is being translated back and forth within the isolated lumen 28 to ablate the lumen having therapeutic agent 80 therein. The ablation implement 70, such as a ablation brush 71 extends radially outward from the ablation conduit 64. The ablation brush may comprise a plurality of individual tines 73 that enable sliding past the isolation balloon inflation conduit 55.

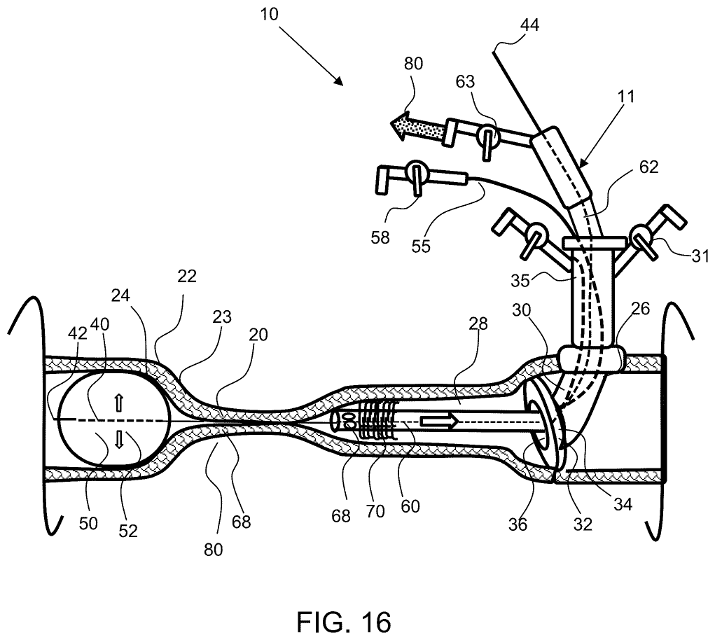

As shown in FIG. 16, the therapeutic agent 80 and any debris is being withdrawn from the isolated lumen 28 by a vacuum drawn through the ablation treatment apparatus, such as though the agent apertures 68 in the conduit wall 65 of the agent conduit 62. The agent apertures 68 may be configured through the ablation conduit wall 65 and on the distal end of the ablation conduit 64 and this may be an effective location to withdraw fluid from the lumen as the ablation implement 70 may prevent the lumen from sealing off the agent apertures. With the agent apertures located just distal the ablation implement 70, the ablation implement, extending radially outward from the ablation conduit wall 65, will prevent sealing off of the agent apertures 68.

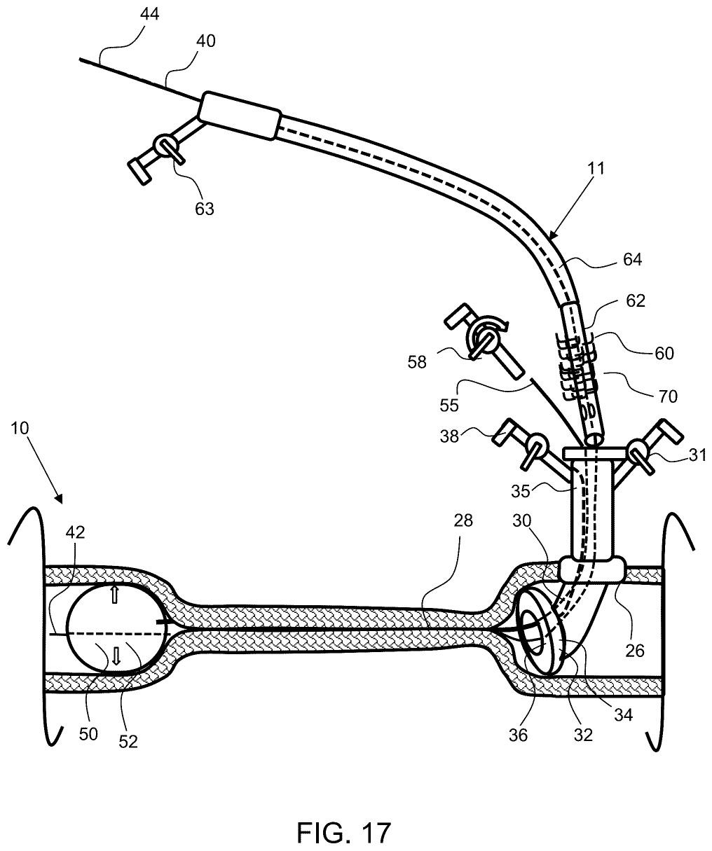

As shown in FIG. 17, the lumen 20 has been collapsed by the vacuum drawn through the ablation treatment apparatus 60. Collapsing the lumen wall may be desired in varicose vein treatment applications. The ablation treatment apparatus 60 has been withdrawn from the isolated lumen through the access-balloon aperture 36 in the access balloon 32 and through the access port 30.

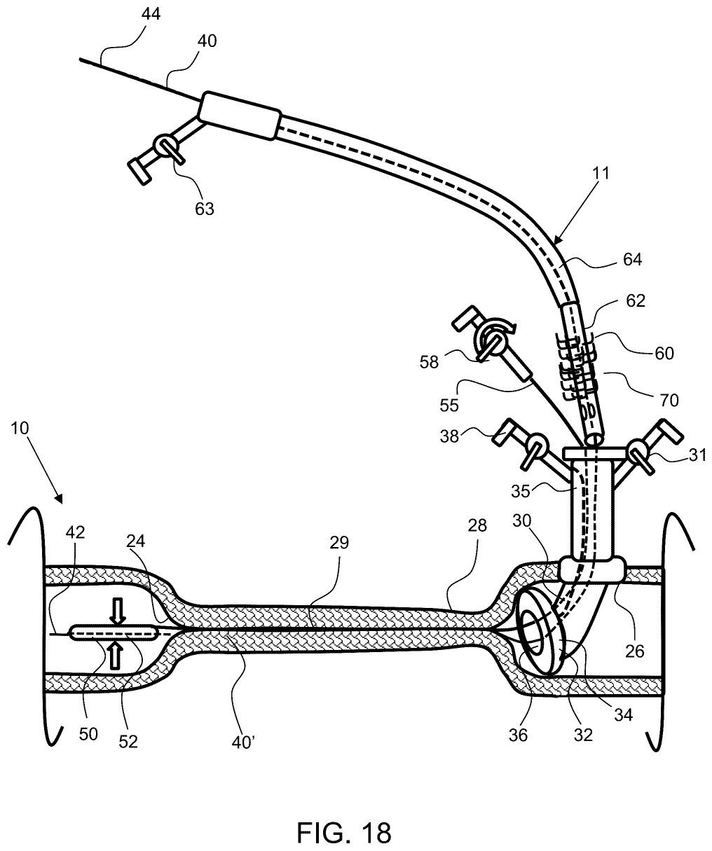

As shown in FIG. 18, the isolation balloon 50 is collapsed for removal.

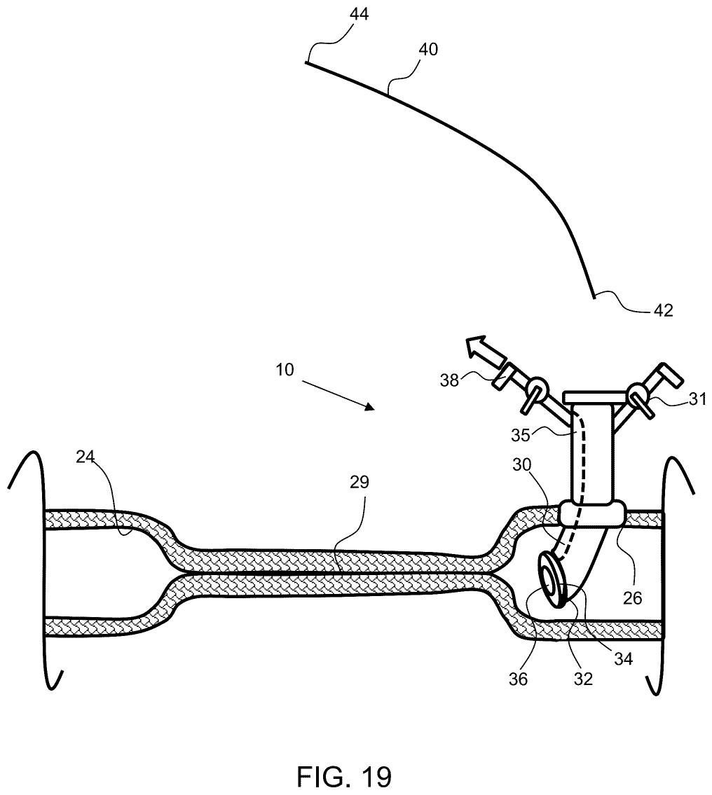

As shown in FIG. 19, the isolation balloon 50 is removed and the isolated lumen 28 is collapsed. Also, the access balloon 32 is deflated for removal of the access port 30 and access balloon 32 through the lumen access aperture 26.

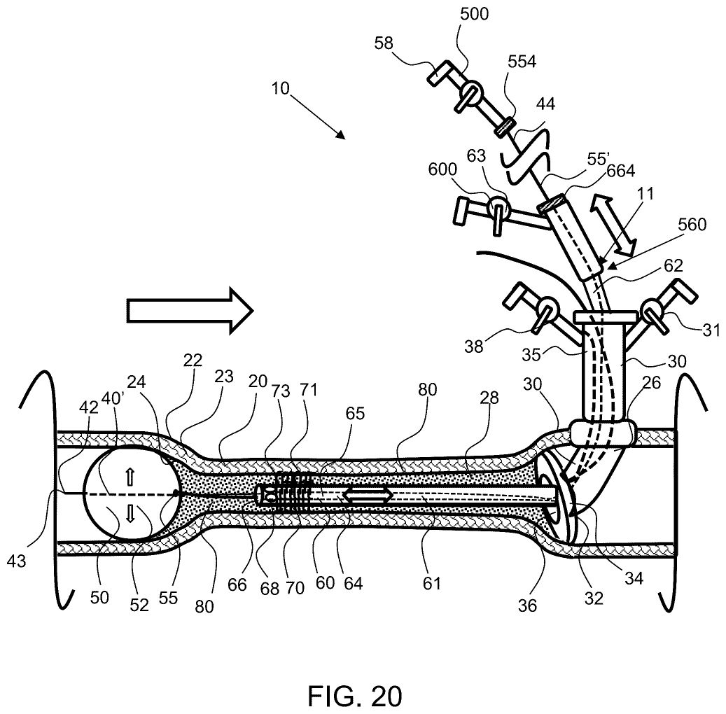

Referring now to FIGS. 20 to 33, a lumen ablation system 10, may utilize an integrated isolation ablation component 560 comprising an isolation balloon assembly 500 with an ablation treatment assembly 600. The integrated isolation ablation component may be inserted through the access port 30 and through the access balloon aperture into the lumen 20. The isolation balloon assembly may be advanced with respect to or with the ablation treatment assembly into the lumen. A balloon lock portion 554 may be configured to lock with an ablation lock portion 664 to affix the two components together, or to release the components so they can be moved independently. As shown in FIG. 20, the isolation balloon 50 is inflated by the isolation balloon inflation conduit 55 and the ablation implement 70 is configured over the isolation balloon inflation conduit. The lock portions are detached so the ablation implement can be moved back and forth over the isolation balloon inflation conduit 55 within the lumen 20. The isolation balloon inflation conduit 55, 55′ extends through the ablation implement and to the isolation balloon interface 58.

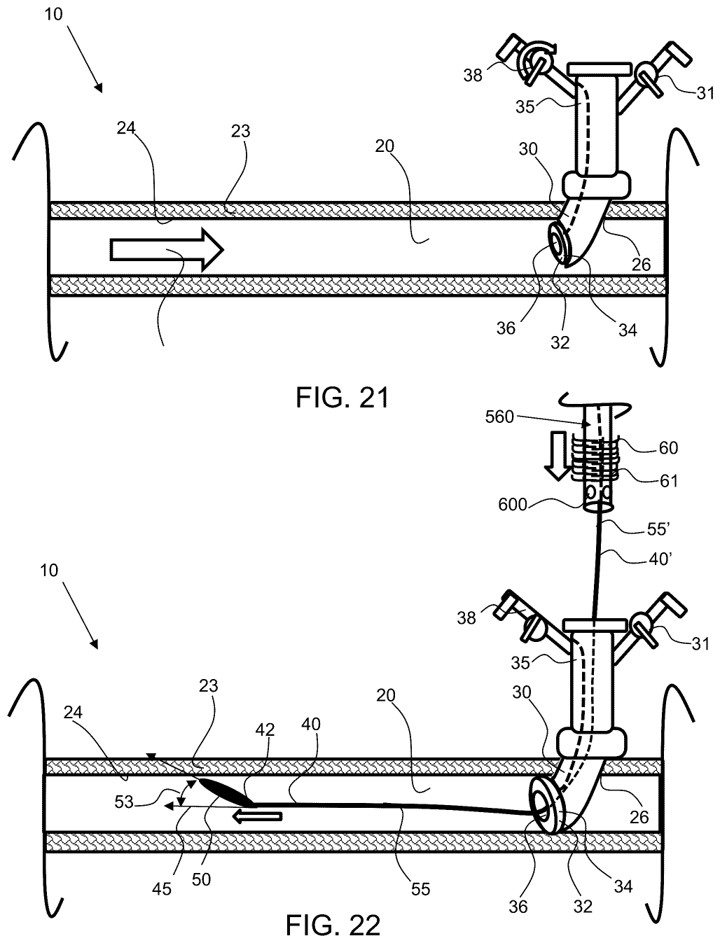

As shown in FIG. 21, an access port 30 is configured in a lumen access aperture 26 in the lumen wall 23 with an access balloon 32, a toroid balloon 34, configured on the distal end of the access port with an access balloon aperture 36 for introducing the isolation balloon inflation conduit 55, that may act as a guide wire, and also the ablation treatment apparatus therethrough. The access balloon 32 is not inflated and does not occlude the lumen 20. The access balloon may be coupled to the access port 30 and the access port may include the access-balloon interface 38. Note that the large arrow in the lumen indicates flow of bodily fluid, such as blood, from a proximal location, proximal the heart, to a distal location, away from the heart.

As shown in FIG. 22, the access balloon 32 has been inflated through the introduction of inflation fluid from the access balloon inflation conduit 35 and the access balloon interface 38. The lumen ablation system 10 has the distal end of the isolation balloon inflation conduit 55, being introduced into the lumen 20 through the access port 30 and the access balloon aperture 36, in the access balloon 32. The isolation balloon 50 is configured on the distal end of the isolation balloon inflation conduit 55 and is configured at an offset angle 53 from the length axis 45 of the guide wire, the isolation balloon inflation conduit 55, to enable the isolation balloon inflation conduit 55 to be steerable within the lumen. Fluoroscopy may be used to determine a position and orientation of the isolation balloon to enable manipulation of the isolation balloon inflation conduit for directing the isolation balloon to a desired location. Note that the ablation treatment apparatus 60 is not yet inserted into the access port 30.

The access balloon has been inflated through the introduction of inflation fluid from the access balloon inflation conduit 35 and the access balloon interface 38. A syringe may be coupled with the access balloon interface to introduce the inflation fluid into the toroid balloon 34 forming the access balloon 32.

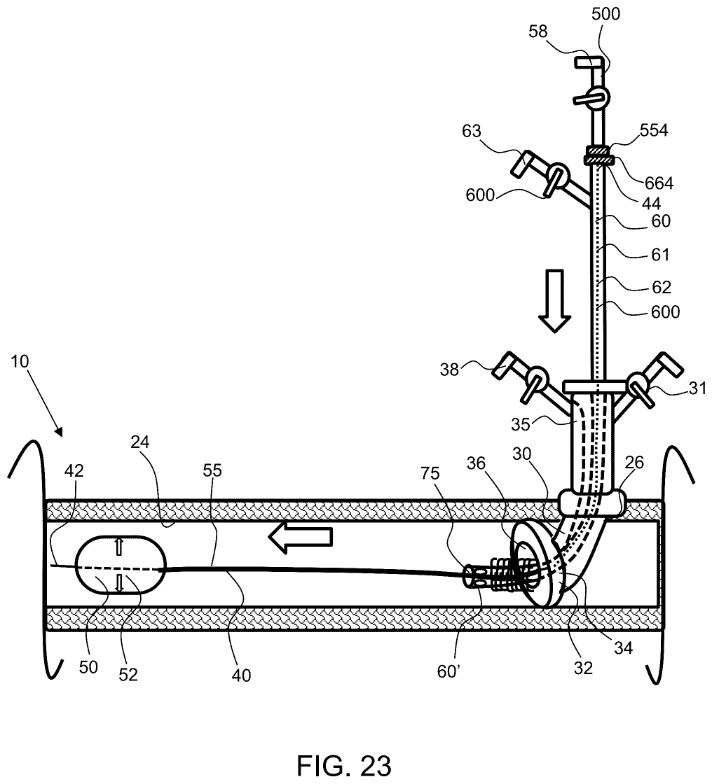

As shown in FIG. 23, the lumen ablation system 10 has the isolation balloon 50 introduced into the lumen 20 through the access port 30, and the access balloon aperture 36 in the access balloon 32. The isolation balloon 50 is being inflated by the introduction of an inflation fluid through the isolation balloon inflation conduit 55 via the isolation balloon interface 58. The isolation balloon inflation conduit 55 may act as guide wire 40. The inflation balloon may be an over-wire balloon that extends over or around the guide wire or may be coupled to the isolation balloon inflation conduit 55. A syringe may be coupled with the isolation balloon interface to inject inflation fluid, such as saline or water, into the isolation balloon 50 to expand the isolation balloon wall 52 against the interior surface 24 of the lumen wall 23. As shown, the ablation treatment assembly 600 is being advanced over the isolation balloon inflation conduit 55 and the ablation treatment apparatus is entering the lumen 20 through the access balloon aperture 36. The ablation treatment apparatus 60 may have an ablation distal end opening 75, such as in the ablation conduit 64 for allowing a flow of therapeutic agent therefrom. Note that the isolation balloon assembly 500 and the ablation treatment assembly 600 may be detachably attached by the isolation lock portion 554 and the ablation lock portion 664. When locked together, the isolation balloon inflation conduit 55 may be advanced by movement of the agent conduit 62 of the ablation treatment apparatus 60.

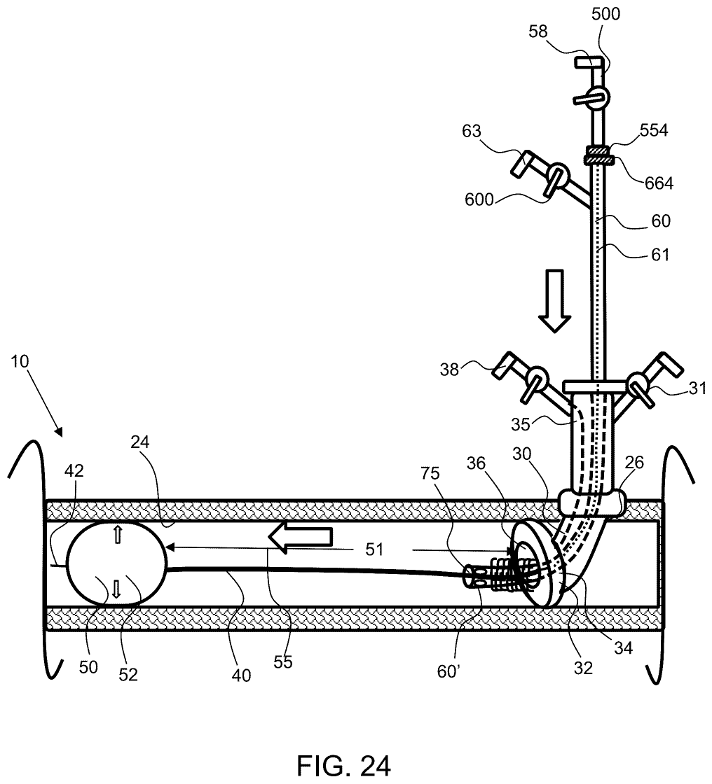

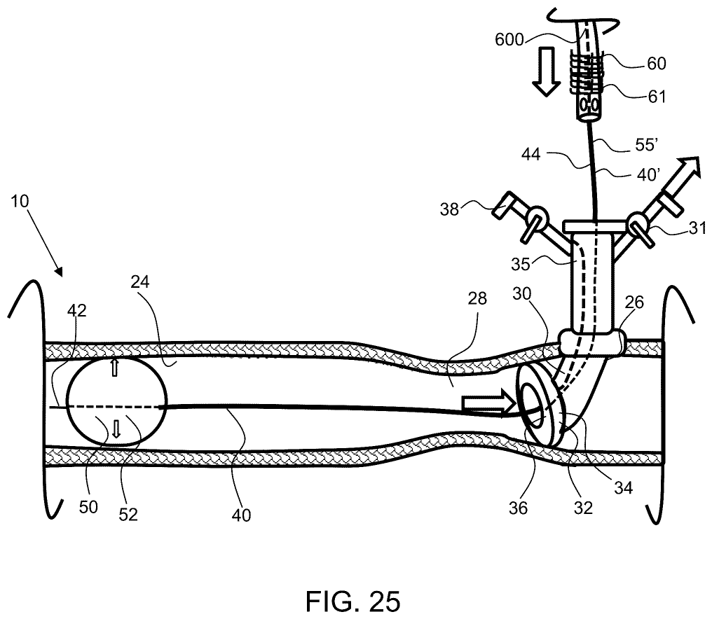

As shown in FIG. 24, the lumen ablation system 10 has the isolation balloon 50 now inflated to produce an isolated lumen 28 having a length 51 between the isolation balloon 50 and the access balloon 32. The isolated lumen will prevent therapeutic agent and thrombus or other debris from the ablation process from traveling along the lumen. The isolation balloon is configured a length 51, or an offset distance from the access balloon 32 to create the isolated lumen 28. The ablation interface may be used to aspirate the lumen by drawing vacuum and pulling out blood or other bodily fluid to collapse the lumen as shown in FIG. 25. Note that the access port 30 may comprise a lumen access interface 31 that may be used as well to aspirate the lumen.

As shown in FIG. 25, the isolated lumen 28 is being aspirated by the lumen access interface 31, as indicated by the large bold arrow extending from the access balloon interface, to remove the blood from the isolated lumen.

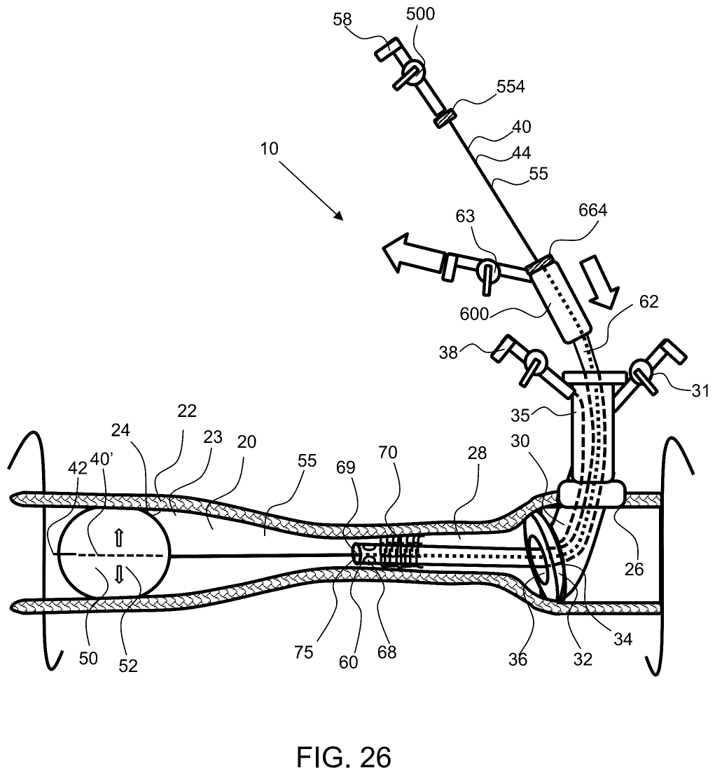

As shown in FIG. 26, the lumen ablation system 10 has the ablation treatment apparatus 60 introduced into the isolated lumen 28 through the access port 30 and through the access balloon aperture 36 in the access balloon 32. The isolated lumen is being further aspirated to remove any additional blood and to collapse the isolated lumen through the agent apertures in the ablation treatment apparatus 60. The ablation treatment apparatus may have an agent aperture on the distal end 69. The ablation treatment apparatus 60 may be moved to a position proximal to the isolation balloon 50 and then withdrawn while the lumen is aspirated to remove blood and collapse the isolated lumen 28. Note that the ablation treatment assembly 600 and isolation balloon assembly 500 are detached by the lock portions of each to allow movement of the ablation treatment apparatus 60 over the isolation balloon inflation conduit 55.

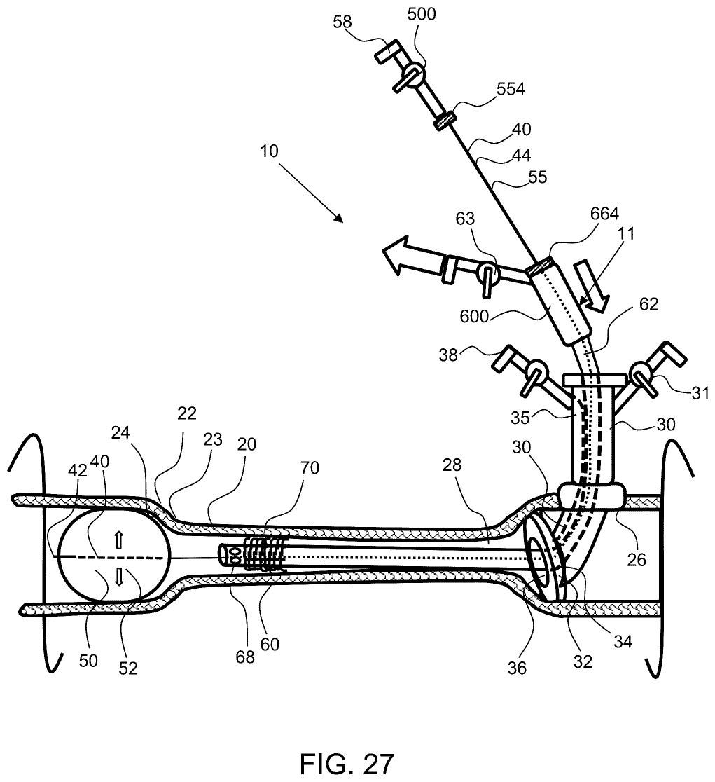

As shown in FIG. 27, the ablation treatment apparatus 60 is translated further toward the isolation balloon 50 and is aspirating the lumen 20 to cause it to collapse down between the isolation balloon and access balloon.

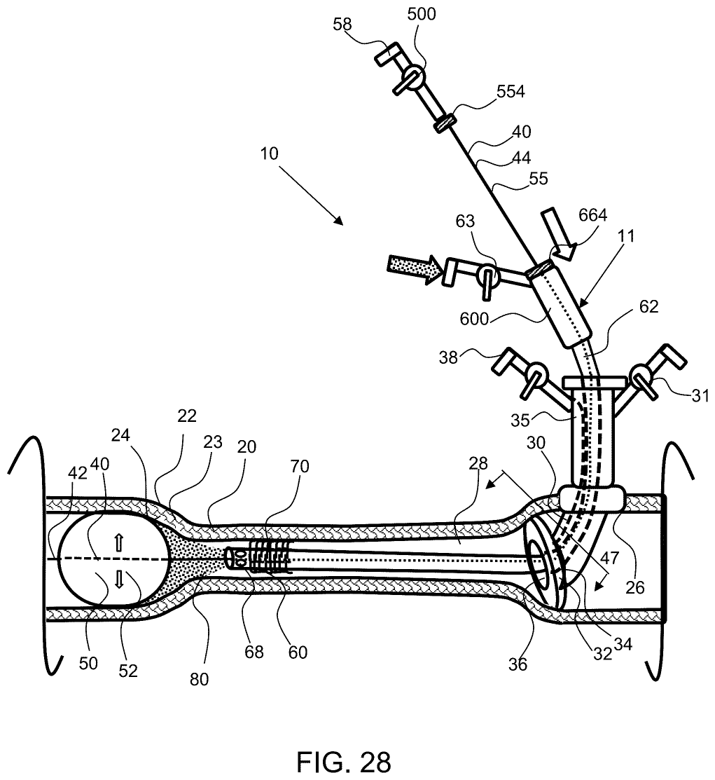

As shown in FIG. 28, therapeutic agent 80 is being introduced into the isolated lumen 28 by the ablation treatment apparatus 60 through the ablation interface 63 as indicated by the large arrow. The therapeutic agent 80 is being dispense from agent apertures 68 in the ablation treatment apparatus 60, such as in the ablation conduit wall 65 or in the distal end of the ablation conduit 64.

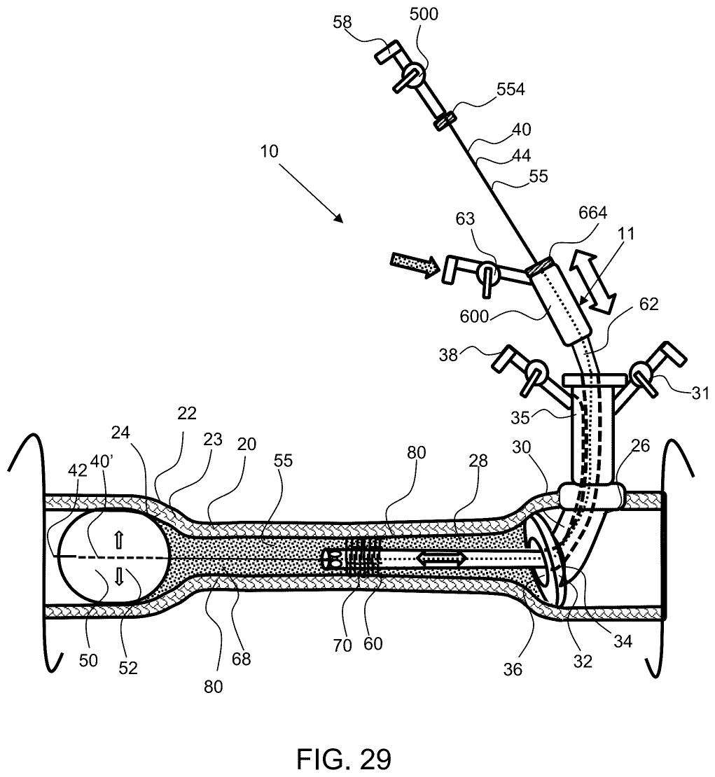

As shown in FIG. 29, the ablation treatment apparatus 60 is being translated back and forth within the isolated lumen 28 to ablate the lumen having therapeutic agent 80 therein. The ablation implement 70, such as a ablation brush 71 extends radially outward from the ablation conduit 64. The ablation brush may comprise a plurality of individual tines 73 or tines that spiral to form radial extensions.

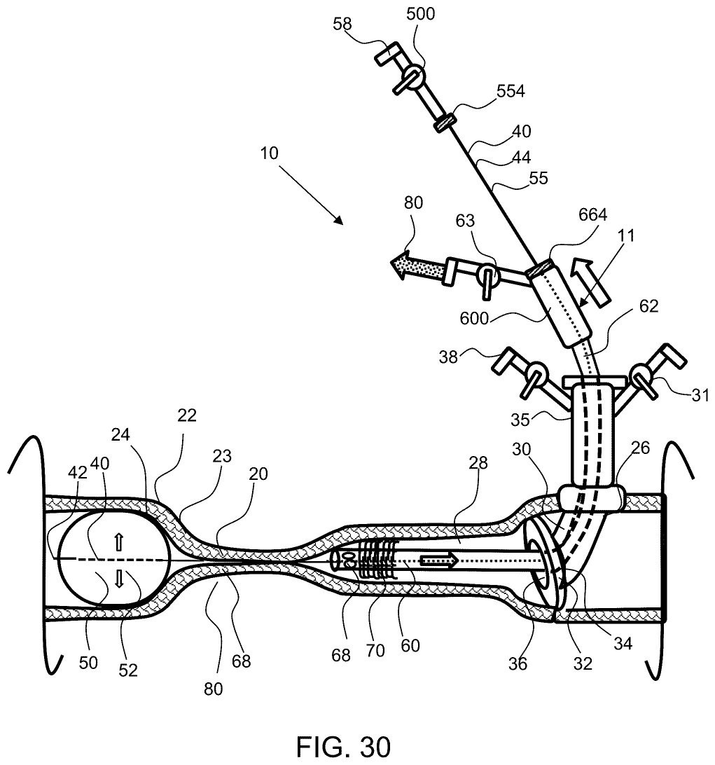

As shown in FIG. 30, the therapeutic agent 80 and any debris is being withdrawn from the isolated lumen 28 by a vacuum drawn through the ablation treatment apparatus, such as though the agent apertures 68 in the conduit wall 65 of the agent conduit 62. The agent apertures 68 may be configured through the ablation conduit wall 65 and on the distal end of the ablation conduit 64 and this may be an effective location to withdraw fluid from the lumen as the ablation implement 70 may prevent the lumen from sealing off the agent apertures. With the agent apertures located just distal the ablation implement 70, the ablation implement, extending radially outward from the ablation conduit wall 65, will prevent sealing off of the agent apertures 68.

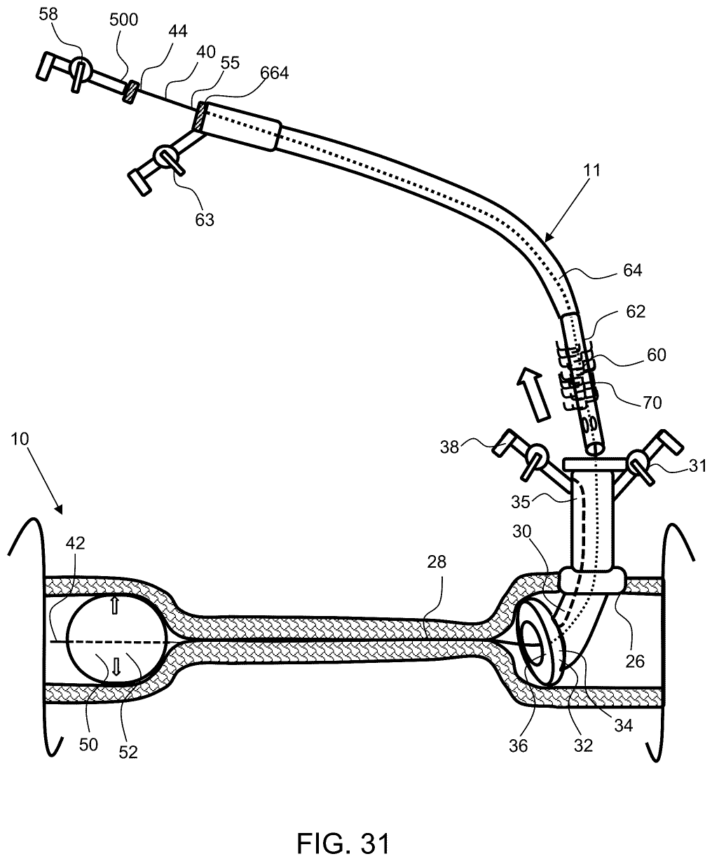

As shown in FIG. 31, the lumen 20 has been collapsed by the vacuum drawn through the ablation treatment apparatus 60. Collapsing the lumen wall may be desired in varicose vein treatment applications. The ablation treatment apparatus 60 has been withdrawn from the isolated lumen through the access-balloon aperture 36 in the access balloon 30 and through the access port 30.

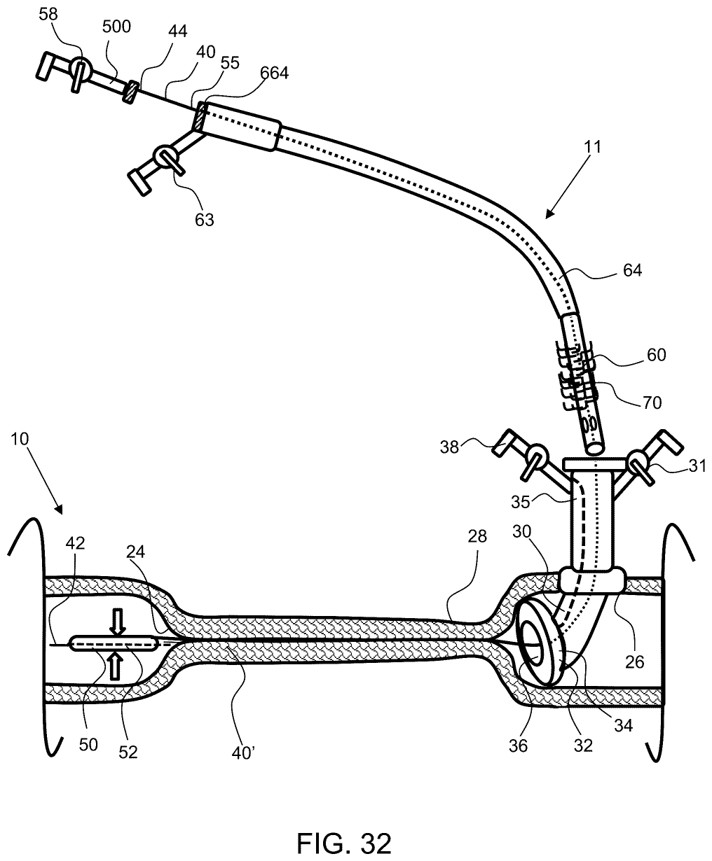

As shown in FIG. 32, the isolation balloon 50 is collapsed for removal.

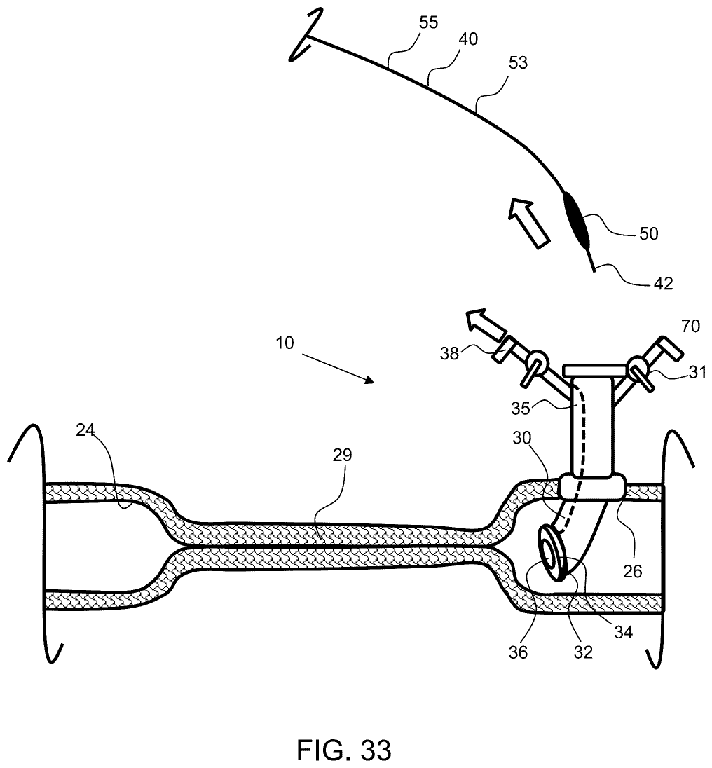

As shown in FIG. 33, the isolation balloon 50 is removed and the isolated lumen 28 is collapsed. Also, the access balloon 32 is deflated for removal of the access port 30 and access balloon 32 through the lumen access aperture 26.

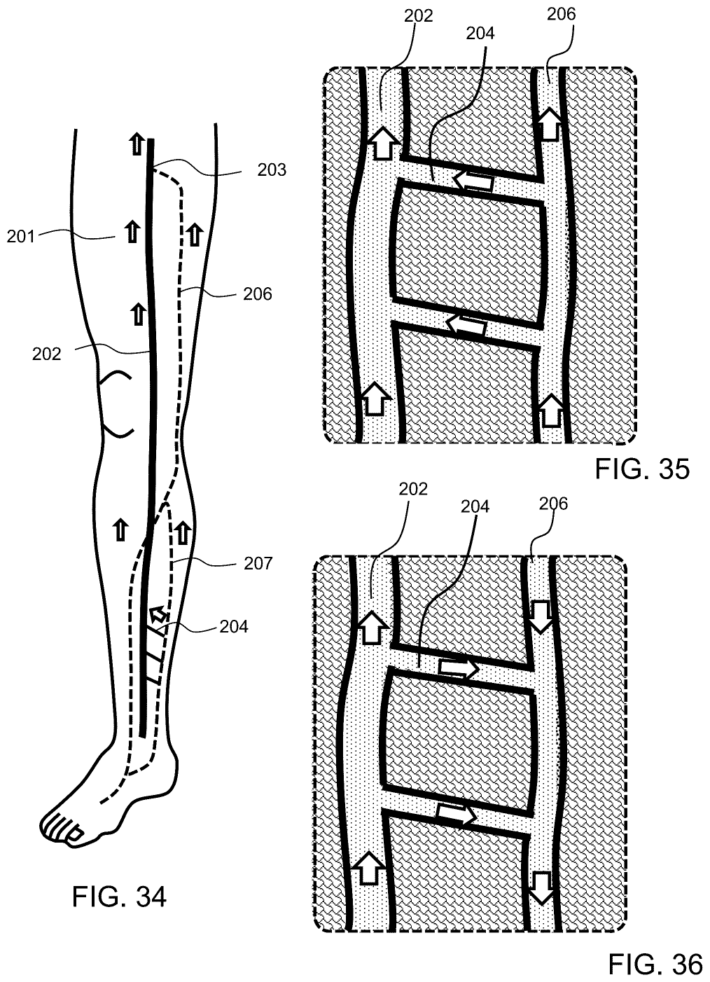

Referring now to FIGS. 34 to 36, the femoral vein 202, the saphenous vein 206 extend along a person's leg 201 and the perforating veins 204 extend between the femoral vein and the saphenous vein. The saphenous vein 206 connects with the femoral vein 202 at the saphenofemoral junction 203. The small saphenous vein 205 branches from the saphenous vein 206.

FIG. 35 shows an expanded view of a healthy vascular system with the blood flow in the femoral vein 202 and saphenous vein 206 flowing from the lower extremity to the heart and blood flow from the saphenous vein through the perforating vein 204 to the femoral vein.

FIG. 36 shows an expanded view of a diseased vascular system with the blood flow in the femoral vein 202 flowing from the lower extremity to the heart but the blood flow in the saphenous vein 206 flowing down toward the lower extremity and blood flow from the femoral vein 202 through the perforating vein 204 to the saphenous vein 206.

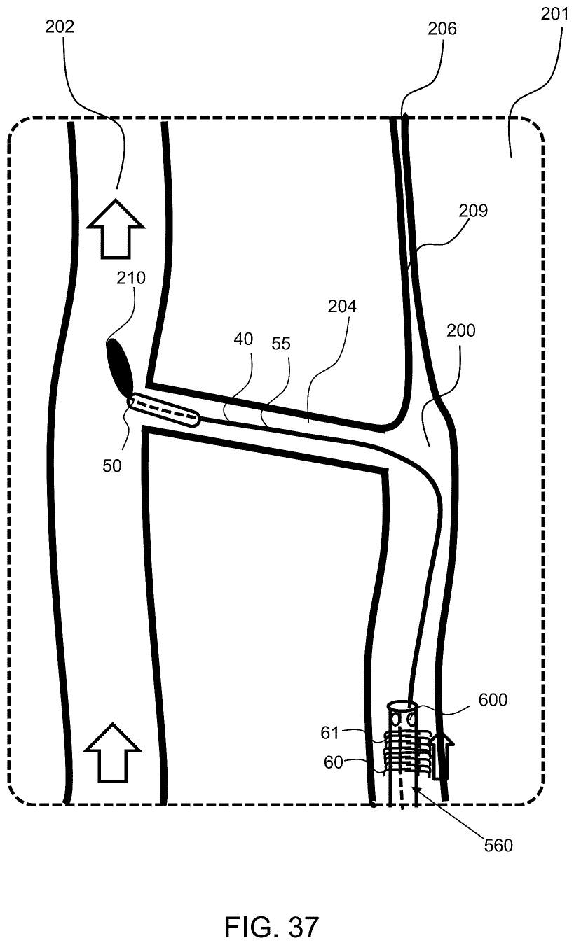

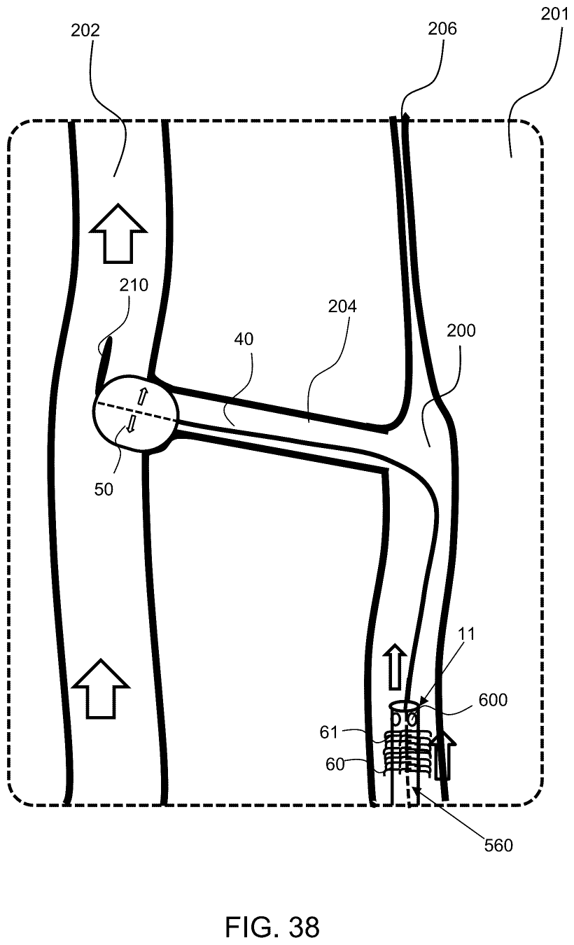

FIG. 37 shows a guide wire being advanced along the saphenous vein 206 and guided through the perforating vein 204 into the femoral vein 202. The isolation balloon inflation conduit 55 may act as the guide wire and have a guide extension 210 on the distal end to steer the isolation balloon inflation conduit 55. The saphenous more proximal to the heart may have been collapsed prior to this view of the procedure to lumen ablate and collapse the perforating vein 204. The saphenous vein 206 more proximal to the heart is a collapsed saphenous vein 209. As described herein, segments of the saphenous vein may be collapsed until the perforating veins are reached. Then the perforating veins may be sequentially lumen ablated and collapsed. As shown, a guide wire 40 is advanced and directed by a guide extension 210, which may be a portion of the isolation balloon 50, into and through the perforating vein 204 and into the femoral vein 202.

As shown in FIG. 28, the isolation balloon 50 is expanded at the intersection of the femoral vein 202 and the perforating vein 204 to seal the perforating vein from the femoral vein. This is done to isolate the perforating vein from the femoral vein to prevent therapeutic agent from flowing into the femoral vein.

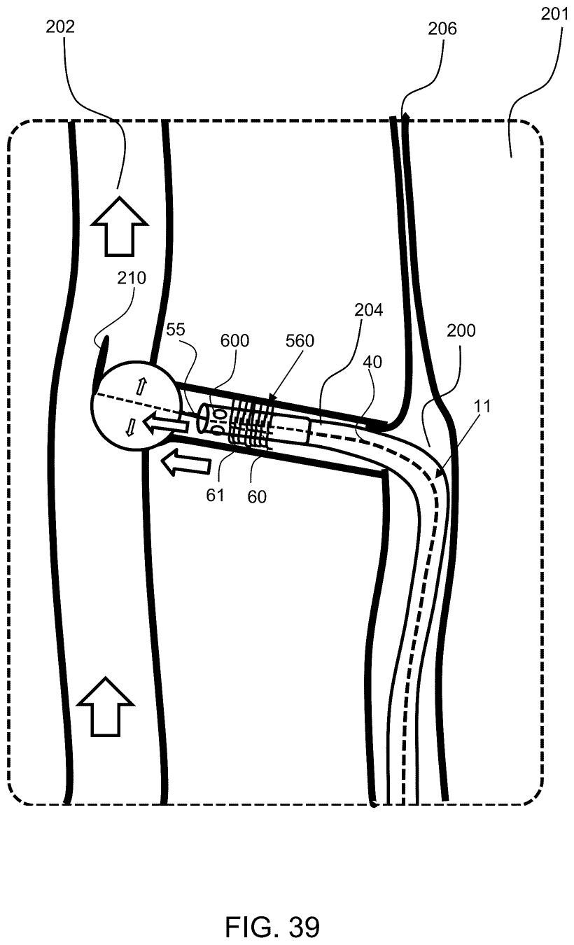

As shown in FIG. 39, the integrated isolation ablation component 560 is advanced over the guide wire 40, isolation balloon inflation conduit 55, into the perforating vein 204. The ablation treatment assembly 600 may be sized to score the interior of the perforating vein 204.

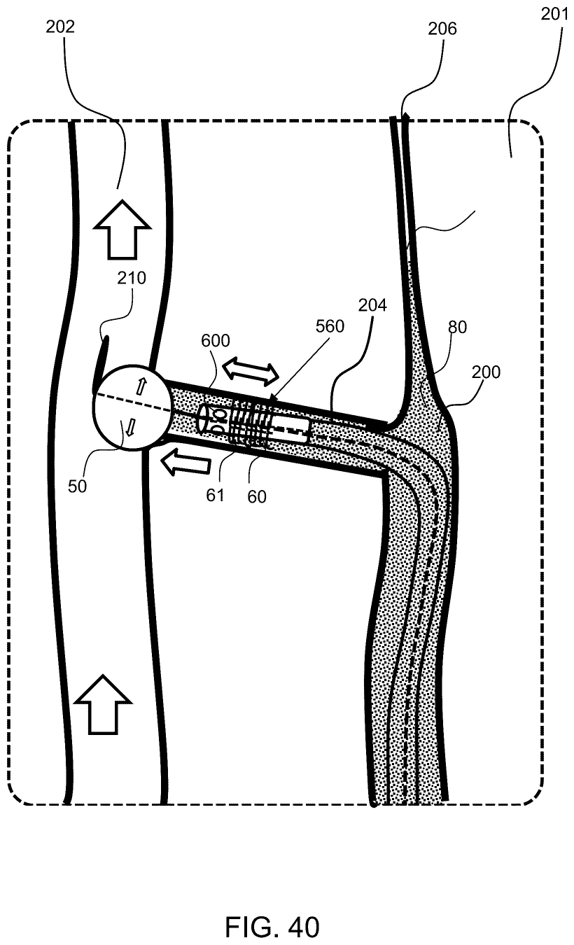

As shown in FIG. 40, therapeutic agent 80 is being dispensed into the perforating vein and the therapeutic agent is being blocked from flow into the femoral vein 202 by the expanded isolation balloon 50. The integrated isolation ablation component 560 may be moved back and forth within the perforating vein 204 to score the interior wall of the perforating vein to ensure collapse of the perforating vein (as indicated by the double ended bold arrow).

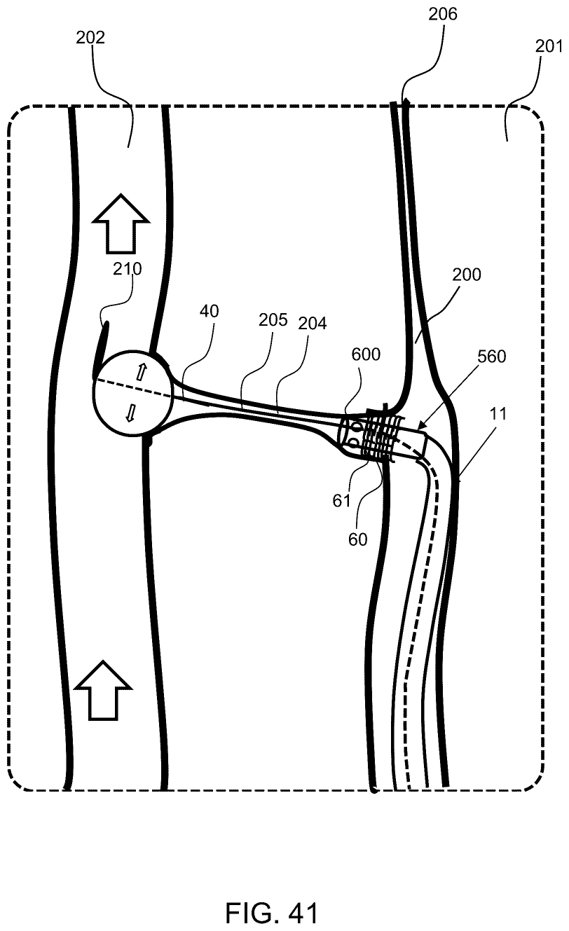

As shown in FIG. 41, the integrated isolation ablation component 560 is retracted from the perforating vein and suction being applied to collapse the perforating vein to produce a collapsed perforating vein 205.

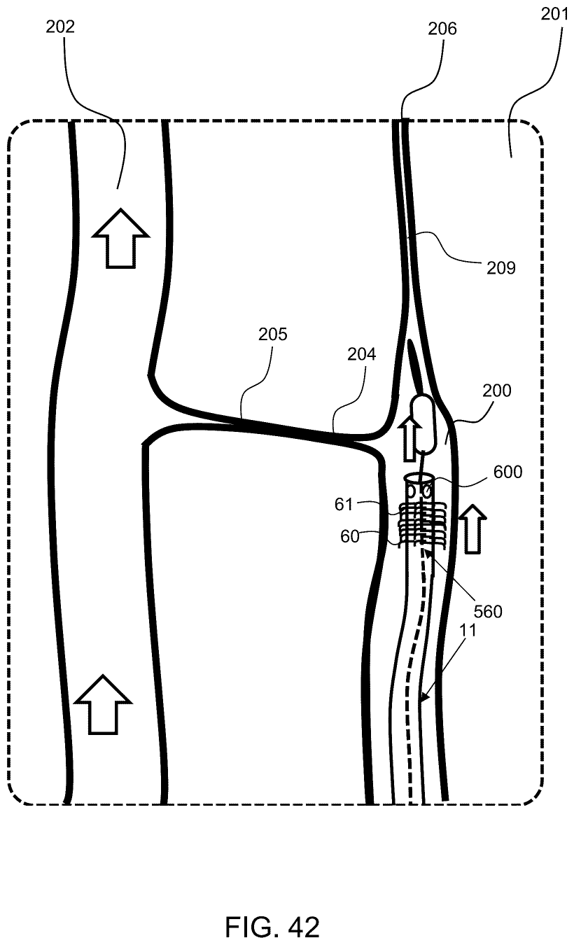

As shown in FIG. 42, the guide wire 40 is now advanced into the saphenous vein to treat and collapse the saphenous vein to the next perforating vein. This procedure can be repeated to collapse a plurality and preferably all of the perforating veins.

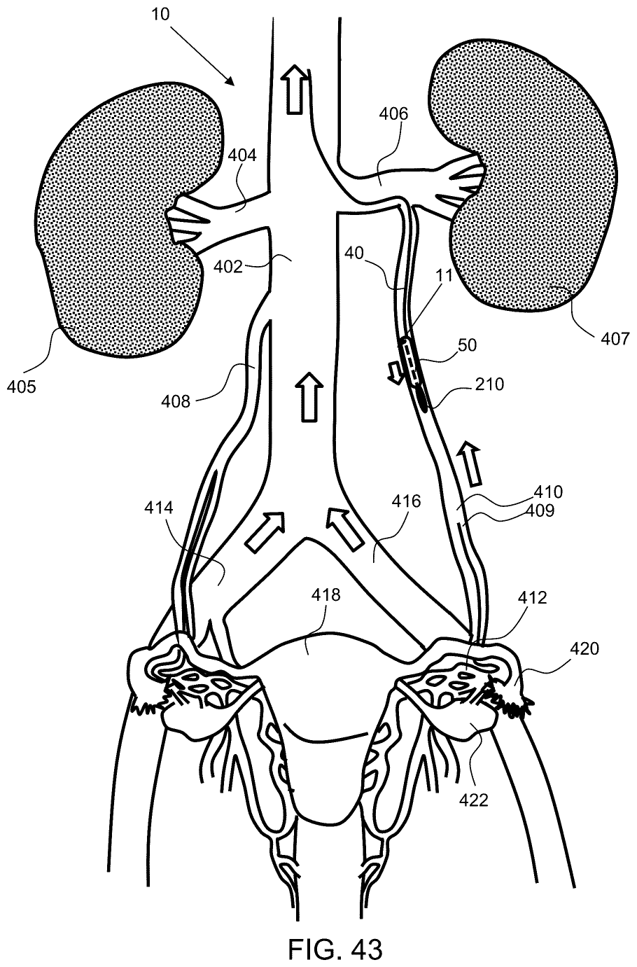

As shown in FIG. 43, blood flow from the uterus 418, ovaries 422 and fallopian tubes 420 may flow through gonadal veins 409, or left and right ovarian veins 410, 408, respectively. A left ovarian vein 410 and right ovarian vein 408 are coupled with a respective ovarian venous plexus 412. A lumen ablation system 10 as described herein, may be used to ablate the left and/or right ovarian vein. As shown, the guide extension 210 has directed the guide wire 40 and isolation balloon 50 into the left ovarian vein 410 from the inferior vena cava, but could be introduced from a renal vein (left renal vein 406) or even along the left ovarian vein 410. The inferior vena cava 402 receives blood from the right common iliac vein 414 and left common iliac vein 416. The right renal vein 404, right kidney 405, left renal vein 406 and left kidney 407 are shown.

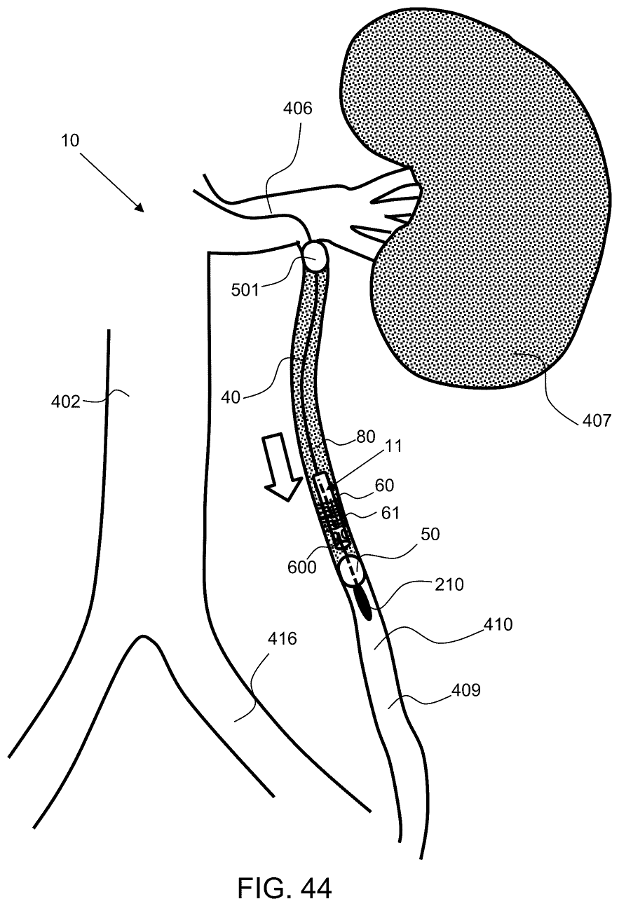

As shown in FIG. 44, a lumen ablation system 10 is being used to ablate the left ovarian vein 410, an example of a gonadal vein 409. In this case, the guide wire 40 is introduced through the left renal vein 406 and may, as described herein, be introduced from the inferior vena cava. The isolation balloon 50 is inflated to stop the flow of blood through the left ovarian vein. A proximal isolation balloon 501 is used to isolate the left ovarian vein 410 and is positioned proximal to the junction of the left ovarian vein and the left renal vein 406. The ablation treatment apparatus 60 has been introduced along the guide wire 40 and is used to ablate the vein. An ablation interface configured outside of the body may be used to introduce a therapeutic agent 80 through the ablation catheter 61. As shown, the therapeutic agent is retained in the left ovarian vein 410 by the isolation balloon 50 and the proximal isolation balloon 501.

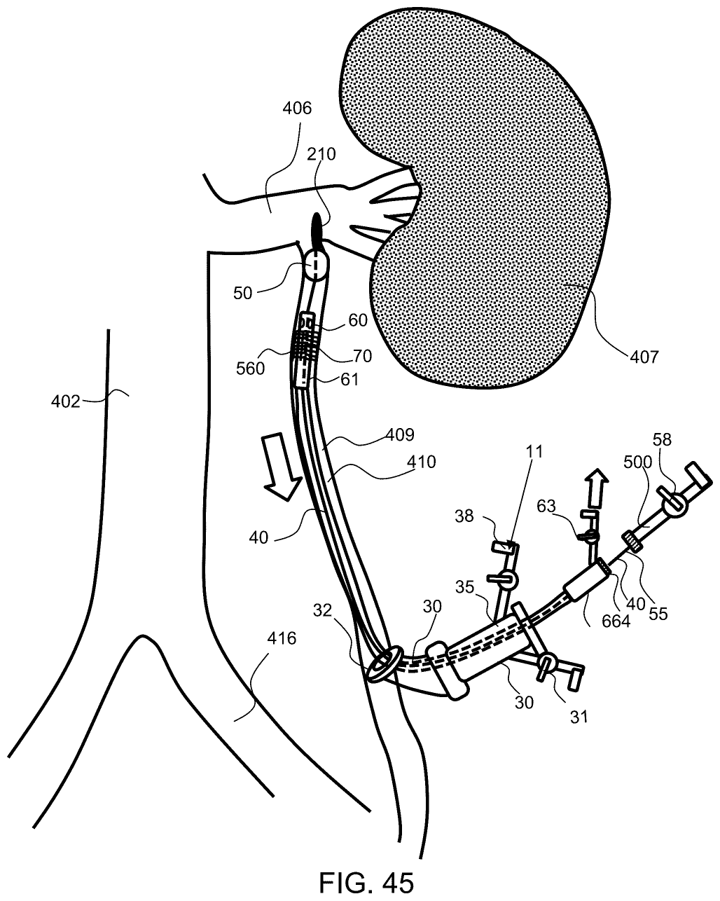

As shown in FIG. 45, a lumen ablation system 10 is being used to ablate the left ovarian vein 410, an example of a gonadal vein 409. In this case, the guide wire 40 is introduced through the left ovarian vein 410 and the isolation balloon is configured proximal to the left renal vein 406. The isolation balloon 50 is inflated to stop the flow of blood through the left ovarian vein. The ablation treatment apparatus 60 has been introduced along the guide wire 40 and is used to ablate the left ovarian vein. Movement of the ablation catheter 61 and the ablation implement 70 along with injection of therapeutic agent into the vein may effectively ablate the vein. An ablation interface configured outside of the body may be used to introduce the therapeutic agent 80 through the ablation catheter 61. As shown, the therapeutic agent is retained in the left ovarian vein 410 by the isolation balloon 50 and the access balloon 32, coupled to the access port 30.

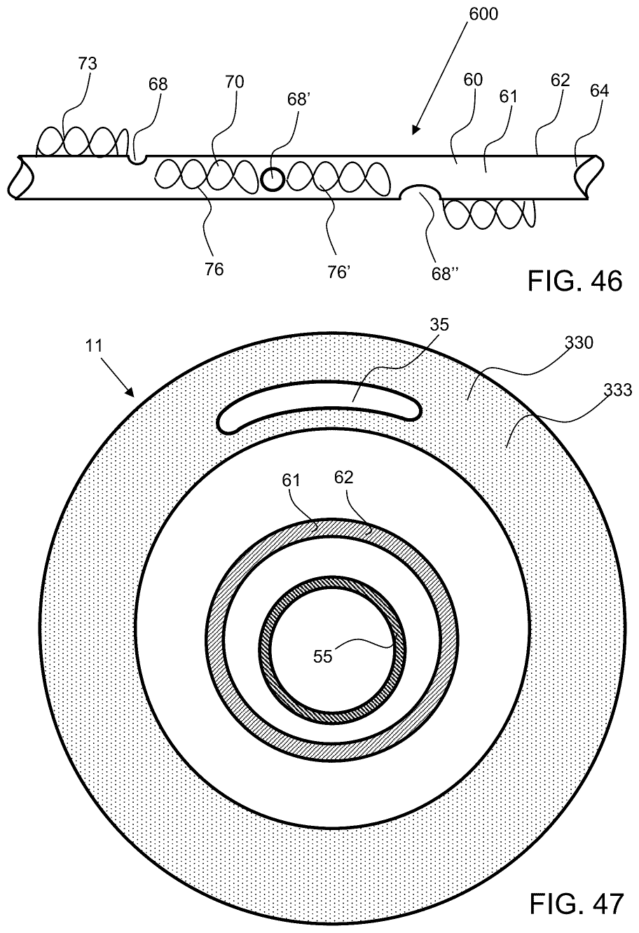

FIG. 46 shows a side view of the ablation treatment assembly 600, including an ablation treatment apparatus 60 having an ablation implement 70 that is configured circumferentially about the ablation catheter 61 and specifically with the ablation implements extensions 76 configured circumferentially aligned with at least one of the agent apertures 68 that are configured on the distal or proximal ends of an ablation implement extension. As shown, ablation implement extensions 76, 76′ are configured circumferentially aligned with agent aperture 68′ and between agent aperture 68′ and 68, and 68′ and 68″, respectively. As shown in FIG. 46 the agent aperture 68′ is configured between ablation implements 70, 70′, or portions, ablation implement extensions 76, 76′. The agent apertures may be configured in alignment with the ablation implements, aligned circumferentially about the ablation conduit 64 to prevent the agent apertures from being sealed off during aspiration of the lumen. An agent aperture may be in alignment about the circumference of the agent conduit 62 or ablation catheter 61 with a portion of the ablation implement 70 or tines 73. Also, the agent apertures may be between the ablation implements, portions or segments of the ablation implements or under the ablation implements radially to prevent sealing of the agent aperture.

FIG. 47 shows a cross-section of the lumen ablation apparatus 11 taken along line 47 of FIG. 27 and shows the access port conduit 333 and access balloon inflation conduit 35, the ablation catheter 61 that forms an agent conduit 62, and the isolation balloon conduit 55 for positioning the isolation balloon and for inflating the isolation balloon, such as with saline. The isolation balloon conduit 55 extends within the agent conduit 62 and the agent conduit extend through the access port conduit 333 or access catheter 330 to form a tri-lumen assembly. As shown, the access balloon inflation conduit 35 is within the wall of the access port conduit 333 but may be a separate conduit extending along the access port conduit to the access balloon.

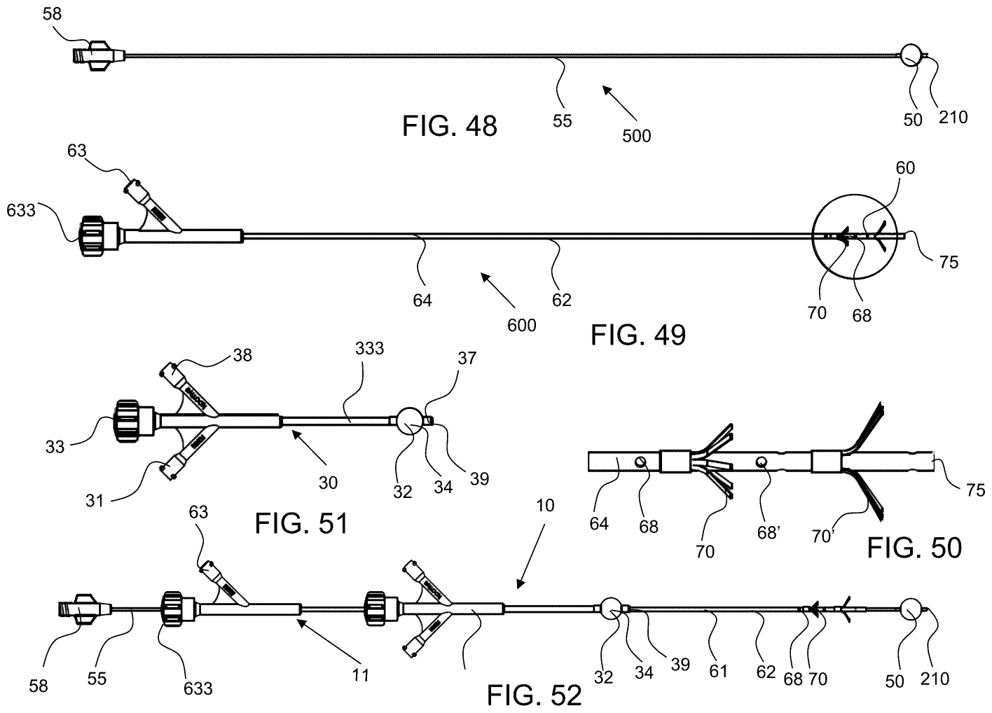

Referring now to FIGS. 48 to 52, a lumen ablation system 10 includes a plurality of components that interface together to enable isolation of a portion of a lumen or vessel between the access balloon and the isolation balloon and also enable ablation and collapsing the lumen within this isolated segment or length of the lumen.

FIG. 48 shows the isolation balloon assembly 500 that is configured to extend through the ablation treatment assembly and through the ablation conduit to locate the isolation balloon distal the ablation treatment assembly. The isolation balloon assembly 500 includes an isolation balloon 50 configured proximal to the distal end of the isolation balloon assembly, and an isolation balloon inflation conduit 55 for inflating the isolation balloon through the isolation balloon interface 58. Also, a guide extension 210 may be configured on the distal end to guide the distal end into a desired lumen, such as into a branched portion of the lumen.

FIG. 49 shows the ablation treatment assembly 600 that is configured to extend into the access port inlet opening 33 and through the access port conduit 333 (shown in FIG. 51) and into a lumen. The ablation treatment assembly 600 has an ablation treatment apparatus 60 with an ablation conduit 64 for receiving therapeutic agent from the ablation interface 63. The isolation balloon assembly 500 (shown in FIG. 48) is configured to extend into the ablation interface inlet opening 633 and the isolation balloon is configured to extend out of the ablation distal end opening 75.

FIG. 50 shows an enlarged view of the ablation treatment apparatus 60 having a plurality of agent apertures 68, 68′ and ablation implements 70, 70′, such as tines 73 extending from the ablation conduit 64 radially outward to ablate an interior of a lumen or vessel wall and wherein the ablation implements may extend in opposing directions about the circumference of the ablation conduit. The agent apertures may be aligned with and between the ablation implement and the ablation conduit 64, wherein the agent apertures are under the ablation implement radially to prevent sealing of the agent apertures during aspiration from the collapsed interior lumen wall. Put another way, the agent apertures are aligned with the ablation implements, aligned along the length of the ablation conduit about the circumference of the ablation conduit 64, as shown in FIG. 46.

FIG. 51 shows the access port 30 having an access port conduit 333 extending from the access port inlet opening 33 to the access port outlet opening 39 and an access balloon 32, a toroid balloon 34, configured proximal to the distal end 37 of the access port and a lumen access interface 31, as well as a access balloon interface 38 for inflating the access balloon.

FIG. 52 shows the lumen ablation system 10 that includes the three components shown in FIGS. 48, 49 and 51 integrated together for isolating a segment of a lumen, such as a vessel, for ablating the vessel and collapsing the vessel. The isolation balloon inflation conduit 55 and isolation balloon 50 of the isolation balloon assembly 500 is inserted through the ablation interface inlet opening 633 and extends through the ablation catheter 61 or agent conduit 62 and out the ablation distal end opening 75. The isolation balloon inflation conduit 55 and isolation balloon 50 of the isolation balloon assembly 500 also extend through the access port 30. The ablation treatment apparatus 60 of the ablation treatment assembly 600 is inserted through the access port inlet opening 33 of the access port 30.

It will be apparent to those skilled in the art that various modifications, combinations and variations can be made in the present invention without departing from the scope of the invention. Specific embodiments, features and elements described herein may be modified, and/or combined in any suitable manner. Thus, it is intended that the present invention cover the modifications, combinations and variations of this invention provided they come within the scope of the appended claims and their equivalents.

What is claimed is:

1. A method of isolating and ablating a lumen comprising:
a) providing a lumen treatment apparatus comprising:
i) an access port configured to extend through a lumen wall and into a lumen, said access port comprising:
a lumen end configured for locating within said lumen; and
an exposed end configured for locating outside of said lumen;
ii) an access balloon interface;
iii) an access balloon attached to the access port and configured to inflate via said access balloon interface within said lumen;
wherein the access balloon is a toroid balloon having an access balloon aperture;
iv) an integrated isolation component comprising:
an isolation balloon assembly comprising:
isolation balloon inflation conduit configured to extend through the access port, through the access balloon aperture and into the lumen to a distal end of the isolation balloon assembly;
an isolation balloon coupled to the isolation balloon inflation conduit and configured to be inflated within the lumen to create an isolated lumen between said isolation balloon and the access balloon;
iv) an ablation treatment assembly comprising:
an ablation treatment apparatus configured to extend through the access port, through the access balloon aperture and into the lumen, said ablation treatment apparatus comprising:
an agent conduit with a plurality of agent apertures configured through the agent conduit an offset distance from an extended end of the agent conduit;
an ablation implement coupled to the agent conduit and extending radially outward from the agent conduit and configured to contact an interior surface of said lumen wall to ablate the lumen wall; and
v) a therapeutic agent configured to be delivered into the lumen through the agent apertures in the agent conduit; and
b) locating the access port through the lumen wall to position said lumen end within said lumen and said exposed end outside of said lumen;
c) inflating the access balloon via the access balloon interface to secure the access port in a fixed position with respect to the lumen;
d) introducing the isolation balloon inflation conduit of the integrated isolation component through access port, into the lumen and moving the distal end of the isolation balloon assembly an offset distance from the access balloon;
e) inflating the isolation balloon via the isolation balloon inflation conduit and the isolation balloon interface to produce an isolation lumen between the isolation balloon and the access balloon;
f) introducing the ablation treatment apparatus through the access port and into the lumen;
g) aspirating the isolated lumen;
h) infusing therapeutic agent into the isolated lumen;
i) ablating the isolated lumen by moving ablation treatment assembly back and forth within the isolated lumen;
j) removing the ablation treatment apparatus from the isolated lumen;
k) deflating the isolation balloon;
l) removing the isolation balloon inflation conduit and isolation balloon from the lumen through the access port;
m) deflating the access balloon; and
n) removing the access port to produce an ablated lumen.

2. The method of claim 1, further comprising aspirating the therapeutic agent from the isolated lumen before deflating the isolation lumen and before deflating the access balloon.

3. The method of claim 1, wherein aspirating the isolated lumen collapses the isolated lumen to reduce a diameter of the lumen.

4. The method of claim 3, further comprising an access balloon inflation conduit that extends from the access-balloon interface to said access balloon.

5. The method of claim 1, wherein the ablation treatment apparatus has an opening in a distal end of the agent conduit.

6. The method of claim 1, wherein the agent apertures are configured in alignment with the ablation implements about a circumference of the agent conduit.

7. The method of claim 6, wherein the agent apertures are configured about a circumference of the agent conduit.

8. The method of claim 7, wherein the ablation implement is configured about the circumference of the agent conduit.

9. The method of claim 8, wherein a portion of the ablation implement is configured in alignment about the circumference of the agent conduit with at least one of the agent apertures.

10. The method of claim 9, wherein the ablation implement comprises a plurality of tines extending radially outward from the agent conduit.

11. The method of claim 1, wherein the ablation treatment assembly further comprises an ablation interface and wherein the agent conduit is coupled to said ablation interface for receiving the therapeutic agent into the agent conduit.

12. The method of claim 1, wherein the ablation treatment apparatus has an ablation distal end opening.

13. The method of claim 12, wherein the isolation balloon inflation conduit extends within the agent conduit and out through the ablation distal end opening of the agent conduit.

14. The method of claim 1, wherein the ablation apparatus is translated within the isolated lumen after the therapeutic agent is introduced into the lumen.

15. The method of claim 1, wherein the lumen is a saphenous vein.

16. The method of claim 1, wherein the lumen is a perforating vein.

17. The method of claim 1, wherein the lumen includes a portion of a saphenous vein and at least a portion of a perforating vein.

18. The method of claim 17, wherein a first perforating vein is ablated and a second perforating vein is ablated by moving the guide wire into a second perforating vein and inflating the isolation balloon.

19. The method of claim 1, wherein the lumen is a gonadal vein.

20. The method of claim 19, wherein the gonadal vein is an ovarian vein.

21. The method of claim 19, wherein the gonadal vein is a testicular vein.

22. The method of claim 19, wherein the isolation conduit is introduced into the gonadal vein from an inferior vena cava.

23. The method of claim 19, wherein the isolation conduit is introduced into the gonadal vein from a renal vein.

24. The method of claim 19, wherein the isolation conduit is introduced into the gonadal vein and wherein the isolation balloon prevents flow into a renal vein.
